(19) United States
(12) Patent Application Publication
(10) Pub. No.: US 2006/0140914 A1
(43) Pub. Date: Jun. 29, 2006

(54) Repairing or replacing tissues or organs

(75) Inventors: Rakesh K Jain, Boston, MA (US); Dai Fukumura, Newton, MA (US)

Correspondence Address: CLARK & ELBING LLP, 101 FEDERAL STREET, BOSTON, MA, 02110, US

(73) Assignee: The General Hospital Corporation, Boston, MA (US)

(21) Appl. No.: 10/533,047

(22) PCT Filed: Oct. 30, 2003
(86) PCT No.: PCT/US03/34838

§ 371 Date: Dec. 14, 2005

Related U.S. Application Data
(60) Provisional application No. 60/422,709, filed on Oct. 31, 2002.

(51) Int. Cl. A61K 35/44

(52) U.S. Cl. 424/93.7

(57) ABSTRACT

In general, this invention provides methods and compositions for stabilizing, repairing, or replacing damaged or diseased tissues or organs by engineering blood vessels in such tissues or organs. In addition, the invention further provides methods and compositions for producing functional microvascular networks useful in tissue engineering.

### STATEMENT AS TO FEDERALLY SPONSORED RESEARCH

This work was supported in part by the National Institutes of Health (PO1-CA-80124-02 and R24-CA-85140-01). The government may have certain rights to this invention.

### BACKGROUND OF THE INVENTION

In general, the invention features methods and compositions for repairing or replacing damaged or diseased tissues or organs.

Approximately eight million surgical procedures are performed annually to treat tissue loss or end-stage organ failure. Although these therapies have saved lives, they have severe limitations. The need for organ transplants exceeds the supply of available organs. In the year 2000, over 50,000 people on the transplantation waiting list failed to receive transplanted organs. Thus, alternatives to traditional transplantation therapies are needed.

Transplantable engineered tissues could be used to address chronic organ shortages if technical limitations could be overcome. The development of clinically transplantable three-dimensional engineered tissues is limited by the fact that tissue assemblies greater than 100-200 μm require a perfused vascular bed to supply nutrients and to remove waste products, metabolic intermediates, and secreted products. Mature functional vascular networks have been difficult to engineer given that vascular development is a complex event involving various cell types and many different growth factors.

During embryonic development, endothelial cells form tubes and connect to form the primary capillary plexus, this process is termed angiogenesis. New vessels are formed by splitting existing vessels in two, or by sprouting from existing vessels. This primary network is remodeled and pruned in a process termed vessel maturation to form distinct microcirculatory units that include capillaries, arteries and veins. VEGF is a growth factor that functions to induce endothelial cell proliferation and sprouting. The nascent vasculature recruits mesodermal cell, such as pericytes and endothelial cells, to surround the developing vasculature; and angiopoietin-1 (Ang-1) is a growth factor that plays an important role in recruiting and regulating the assembly of non-endothelial vessel wall components. The interaction between endothelial cells and pericytes leads to the reorganization of vessels into arteries, arterioles, capillaries, venules, or veins. TGF-β, a growth factor that inhibits endothelial cell proliferation and migration, induces mesodermal cell differentiation, and stabilizes the mature capillary network.

### SUMMARY OF THE INVENTION

The present invention features methods and compositions for promoting blood vessel formation or engineering blood vessels in damaged, diseased, or transplanted organs, and for producing functional microvascular networks useful in tissue engineering.

In one such aspect, the invention provides a method for inducing blood vessel formation (e.g., angiogenesis, vasculogenesis, formation of an immature blood vessel network, blood vessel remodeling, blood vessel stabilization, blood vessel maturation, blood vessel differentiation, or establishment of a functional blood vessel network) or engineering blood vessels in a mammal (e.g., a human). The method involves administering one or more cells including, but not limited to, perivascular cells, vascular smooth muscle cells, mesenchymal precursor cells (e.g., 10T1/2 cells), mesenchymal cells, fibroblasts, adipocytes having or not having a genetic modification, preadipocytes (e.g., 3T3-F442A cells), or stem cells that differentiate into one of these cell types to a tissue or organ of a mammal in need of increased blood vessel formation or engineered blood vessels (e.g., a mammal with a damaged or diseased tissue or organ, or a mammal requiring a transplant). In one preferred embodiment, the mammal has a deficiency of at least 5%, 10%, 25%, 50%, 75%, 90% or more of a particular cell type. In another preferred embodiment, the mammal has damage to a tissue or organ, and the method provides a dose of cells sufficient to increase a biological function of the tissue or organ by at least 5%, 10%, 25%, 50%, 75%, 90%, 100%, or 200%, or even by as much as 300%, 400%, or 500%. In yet another preferred embodiment, the mammal has a disease, disorder, or condition, and the method provides a dose of cells sufficient to ameliorate or stabilize the disease, disorder, or condition. For example, the mammal may have a disease, disorder, or condition that results in the loss, atrophy, dysfunction, or death of cells. Exemplary treated conditions include a neural, glial, or muscle degenerative disorder, muscular atrophy or dystrophy, heart disease such as congenital heart failure, hepatitis or cirrhosis of the liver, an autoimmune disorder, diabetes, cancer, a congenital defect that results in the absence of a tissue or organ, or a disease, disorder, or condition that requires the removal of a tissue or organ, ischemic diseases such as angina pectoris, myocardial infarction and ischemic limb, accidental tissue defect or damage such as fracture or wound. In other embodiments, the mammal has an increased risk of developing a disease, disorder, or condition that is delayed or prevented by the method.

In a related aspect, the invention features a method for increasing blood vessel formation or engineering blood vessels in a tissue or organ (e.g., a human tissue or organ) by administering preadipocytes, adipocytes having or not having a genetic modification, perivascular cells, vascular smooth muscle cells, mesenchymal precursor cells (e.g., 10T1/2 cells), mesenchymal cells, or fibroblasts to a tissue or organ in need of increased blood vessel formation or an engineered blood vessel network.

In preferred embodiments of any of the above aspects, the tissue or organ may be in vivo or ex vivo. In various preferred embodiments, the tissue or organ is selected from the group consisting of bladder, brain, nervous tissue, glia, esophagus, fallopian tube, heart, pancreas, intestines, gall bladder, kidney, liver, lung, ovaries, prostate, spinal cord, spleen, stomach, testes, thymus, thyroid, trachea, urogenital tract, ureter, urethra, uterus, breast, skeletal muscle, skin, bone, and cartilage. In other preferred embodiments, the administered cells are from the same mammal. In other embodiments, the tissue or organ is from a different mammal than the administered cells. Preferably, the method further includes administering the tissue or organ to a recipient mammal. The tissue or organ may be from a donor of the same species as the recipient or from a different species (e.g., pig or primate). Preferably, the administration of cells increases the biological function of a diseased or damaged tissue or organ by at least 5%, 10%, 25%, 50%, 75%, 100%, 200%, or even by as much as 300%, 400%, or 500%.

In one preferred embodiment of any of the above aspects, the method further involves administering to the tissue or organ one or more cells selected from the group consisting of blood vascular endothelial cells, lymph vascular endothelial cells, endothelial cell lines, primary culture endothelial cells, endothelial cells derived from stem cells, bone marrow derived stem cells, cord blood derived cells, human umbilical vein endothelial cells (HUVEC), lymphatic endothelial cells, endothelial pregenitor cells, and stem cells that differentiate into endothelial cells, endothelial cell lines, or endothelial cells generated from stem cells in vitro. In preferred embodiments the cell is a HUVEC cell. Preferably, the method further involves administering a matrix to the tissue, organ, or mammal. A matrix may be composed of any biocompatible material, such as synthetic polymers or hydrogels. For some applications, biodegradable materials are particularly desirable. Preferred matrix components include collagen and fibronectin.

In other preferred embodiments, the method increases the number of cells of the tissue or organ by at least 5%, 10%, 20%, more desirably by at least 25%, 30%, 35%, 40%, 50%, 60%, or even by as much as 70%, 80%, 90 or 100% compared to a corresponding tissue or organ. In preferred embodiments, the method increases the biological activity of the tissue or organ by at least 5%, 10%, 20%, 30%, 40%, 50%, 60%, 70%, 80%, 90%, 100%, 150%, or even by as much as 200%, 300%, 400%, or 500% compared to a corresponding, naturally-occurring tissue or organ.

In other preferred embodiments of the above aspects, the method increases blood vessel formation (e.g., angiogenesis, vasculogenesis, formation of an immature blood vessel network, blood vessel remodeling, blood vessel stabilization, blood vessel maturation, blood vessel differentiation, or establishment of a functional blood vessel network) in the tissue or organ by at least 5%, 10%, 20%, 25%, 30%, 40%, or 50%, 60%, 70%, 80%, 90%, or even by as much as 100%, 150%, or 200% compared to a corresponding, naturally-occurring tissue or organ. In other preferred embodiments, the tissue or organ is selected from the group consisting of bladder, bone, brain, breast, cartilage, nervous tissue, glia, esophagus, fallopian tube, heart, pancreas, intestines, gallbladder, kidney, liver, lung, ovaries, prostate, spinal cord, spleen, skeletal muscle, skin, stomach, testes, thymus, thyroid, trachea, urogenital tract, ureter, urethra, and uterus.

In another aspect, the invention provides a method for transplanting a tissue or organ in a mammal (e.g., a human). The method involves administering to the mammal a tissue or organ having at least 5%, 10%, 20%, 40%, 60%, 80%, 100%, 150%, 200%, 300%, 400%, or 500% more perivascular cells, vascular smooth muscle cells, mesenchymal precursor cells (e.g., 10T1/2 cells or embryonic stem cells), mesenchymal cells, preadipocytes (e.g., 3T3-F442A cells), adipocytes not having a genetic modification, or fibroblasts, than a corresponding naturally-occurring tissue or organ. In one preferred embodiment, the method further involves administering to the mammal one or more blood vascular endothelial cells (e.g., HUVEC cells), lymph vascular endothelial cells, or endothelial cell lines, freshly prepared primary culture endothelial cells (e.g., cells obtained from the donor or recipient mammal), or endothelial cells generated from stem cells in vitro. The administered cells may be from the recipient mammal or from another mammal. In other preferred embodiments of the above aspects, the cells are part of a microvascular scaffold or a perfused microvascular scaffold.

In one preferred embodiment, the method further involves administering to the mammal a matrix. A matrix may be composed of any biocompatible material, such as synthetic polymers or hydrogels. Preferred matrices are biodegradable. Preferred matrix components include collagen and fibronectin.

The invention also provides methods to form a microvascular scaffold by incubating at least two cell types (e.g., endothelial cells or endothelial precursor cells and cells of at least one other cell type) under conditions that produce a network of blood vessels. Preferably, the blood vessels grow throughout a cultured tissue. The microvascular scaffolds of the invention can be produced in vivo or ex vivo. The microvascular scaffolds can be administered to mammals to repair or replace an endogenous tissue or organ.

In one such aspect, the invention provides a method for producing a microvascular scaffold. The method involves culturing (i) a first cell selected from the group consisting of blood vascular endothelial cells (e.g., HUVEC cells), lymph vascular endothelial cells, and endothelial cell lines; and (ii) a second cell selected from the group consisting of perivascular cells, vascular smooth muscle cells, mesenchymal precursor cells (e.g., 10T1/2 cells), mesenchymal cells, preadipocytes (e.g., 3T3-F442A cells), adipocytes, and fibroblasts, under conditions that allow formation of a microvascular scaffold. In one preferred embodiment, the first and second cells are cultured in the presence of a matrix. In another preferred embodiment, the cells are grown on the matrix. In another preferred embodiment, the matrix encapsulates the cells. Optionally, the method further involves administering a matrix to a mammal. In one preferred embodiment, cells are present in the matrix prior to, during, or after the matrix is administered to the mammal.

The matrix may be composed of any biocompatible material (e.g., synthetic polymers or hydrogels). For some applications, biodegradable materials are particularly desirable. Preferably, the microvascular scaffold is a stable network of blood vessels that endures for at least 1 day, 2 days, 3 days, 1 week, 2 weeks, 3 weeks, 1 month, 3 months, 6 months, or even as long as 12 months or more. In one preferred embodiment, the microvascular scaffold is a perfused scaffold that is integrated into the circulatory system of the tissue, organ, or mammal. In another preferred embodiment, the microvascular scaffold is a mature network of differentiated vessels that includes arterioles and venules.

In a related aspect, the invention features a microvascular scaffold that includes (i) a first cell selected from the group consisting of blood vascular endothelial cells (e.g., HUVEC cells), lymph vascular endothelial cells, and endothelial cell lines; and (ii) a second cell selected from the group consisting of perivascular cells, vascular smooth muscle cells, mesenchymal precursor cells (e.g., 10T1/2 cells), adipocytes, and fibroblasts. In one embodiment, the microvascular scaffold persists for at least 1 day, 2 days, 3 days, 1 week, 2 weeks, 3 weeks, 1 month, 3 months, 6 months, or even, most preferably, as long as 12 months or more.

In a another related aspect, the invention features a perfused microvascular scaffold that includes (i) a first cell selected from the group consisting of blood vascular endothelial cells (e.g., HUVEC cells), lymph vascular endothelial cells, and endothelial cell lines; and (ii) a second cell selected from the group consisting of mesenchymal precursor cells (e.g., embryonic stem cells or 10T1/2 cells) and mesenchymal cells.

In preferred embodiments of the above aspects, the microvascular scaffold further includes a matrix. A matrix may be composed of any biocompatible material, such as synthetic polymers or hydrogels. Preferred matrices are biodegradable. Preferred matrix components include collagen and fibronectin.

In preferred embodiments of the above aspects, the miscrovascular scaffold further comprises a bioactive molecule. The cells of the scaffold can be genetically engineered to express the bioactive molecule or the bioactive molecule can be added to the matrix, if present. The scaffold can also be cultured in the presence of the bioactive molecule. The bioactive molecule can be added prior to, during, or after culturing the cells to produce a microvascular scaffold. In one example, the bioactive molecule is added to the cell culture media. In another example, the bioactive molecule is delivered locally to the scaffold by direct injection or by a controlled release mechanism such as a pump.

Non-limiting examples of bioactive molecules include activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin, fibronectin receptor α5β1, Factor X, HB-EGF, HBNF, HGF, HUAF, heart derived inhibitor of vascular cell proliferation, IFN-gamma, IL1, IGF-2 IFN-gamma, integrin receptors, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPARγ, PPARγ ligands, phosphodiesterase, prolactin, prostacyclin, protein S, smooth muscle cell-derived growth factor, smooth muscle cell-derived migration factor, sphingosine-1-phosphate-1 (S1P1), Syk, SLP76, tachykinins, TGF-beta, Tie 1, Tie2, TGF-β, and TGF-β receptors, TIMPs, TNF-alpha, TNF-beta, transferrin, thrombospondin, urokinase, VEGF-A, VEGF-B, VEGF-C, VEGF-D, VEGF-E, VEGF, VEGF₁₆₄, VEGI, EG-VEGF, VEGF receptors, PF4, 16 kDa fragment of prolactin, prostaglandins E1 and E2, steroids, heparin, 1-butyryl glycerol (monobutyrin), and nicotinic amide. In other preferred embodiments, the matrix includes a chemotherapeutic agent or immunomodulatory molecule. Such agents and molecules are known to the skilled artisan.

In other preferred embodiments of the above aspects, the interstices of the scaffold include one or more additional cell-types including, but not limited to, skin cells, liver cells, heart cells, kidney cells, pancreatic cells, lung cells, bladder cells, stomach cells, intestinal cells, cells of the urogenital tract, breast cells, skeletal muscle cells, skin cells, bone cells, cartilage cells, keratinocytes, hepatocytes, gastro-intestinal cells, epithelial cells, endothelial cells, mammary cells, skeletal muscle cells, smooth muscle cells, parenchymal cells, osteoclasts, or chondrocytes. These cell-types may be introduced prior to, during, or after microvascular scaffold formation. This introduction may take place in vitro or in vivo. When the cells are introduced in vivo, the introduction may be at the site of the microvascular scaffold or at a site removed from the microvascular scaffold. Exemplary routes of administration of the cells include injection and surgical implantation.

In preferred embodiments of the above aspects, the first or second cells of the microvascular scaffold are genetically modified to expresses a fluorescent protein marker. Exemplary markers include GFP, EGFP, BFP, CFP, YFP, and RFP.

In another aspect, the invention features a tissue or organ that contains a microvascular scaffold. The tissue can also contain one or more additional cell-types, including, but not limited to, cells derived from bladder, brain, nervous tissue, esophagus, fallopian tube, glia, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, or cartilage.

In another aspect, the invention provides for a method of repairing a diseased or damaged tissue or organ. The method involves implanting one or more cells selected from the group consisting of perivascular cells, vascular smooth muscle cells, mesenchymal precursor cells, mesenchymal cells, preadipocytes, adipocytes, and fibroblasts. In one embodiment, the implanted cells increase blood vessel formation (e.g., angiogenesis, vasculogenesis, formation of an immature blood vessel network, blood vessel remodeling, blood vessel stabilization, blood vessel maturation, blood vessel differentiation, or establishment of a functional blood vessel network) or increase the function of a blood vessel network by at least 5%, 10%, 20%, 30%, 50%, 60%, 75%, 80%, 90%, or even by as much as 100%, 150%, or 200% in the damaged tissue or organ as compared to a naturally-occurring, corresponding tissue or organ. In a preferred embodiment, the implanted cells improve the biological function of the diseased or damaged organ by at least 5%, 10%, 20%, 30%, 50%, 60%, 75%, 80%, 90%, 100%, 200%, or even by as much as 300%, 400%, or 500% compared to a naturally-occurring, corresponding tissue or organ. In another preferred embodiment, the implanted cells increase cell number in the diseased or damaged organ by at least 5%, 10%, 20%, 30%, 50%, 60%, 75%, 80%, 90%, or 95% as compared to a naturally-occurring, corresponding tissue or organ.

In a related aspect, the invention features a tissue or organ having at least 5%, 10%, 20%, 40%, 60%, 80%, 100%, 150%, 200%, 300%, 400%, or 500% more of an implanted cell-type selected from the group consisting of perivascular cells vascular smooth muscle cells, mesenchymal precursor cells, mesenchymal cells, preadipocytes, adipocytes, and fibroblasts, than a corresponding naturally-occurring tissue, or organ. In one preferred embodiment, the tissue or organ further contains a transplanted cell selected from the group consisting of blood vascular endothelial cells, lymph vascular endothelial cells, and endothelial cell lines. In another preferred embodiment, the tissue or organ has at least a 5%, 10%, or 20%, 30%, 40%, 50%, 60%, 75%, 80%, 90%, 100%, 200%, or even 300%, 400%, or 500% increase in blood vessel formation (e.g., angiogenesis, vasculogenesis, formation of an immature blood vessel network, blood vessel remodeling, blood vessel stabilization, blood vessel maturation, blood vessel differentiation, or establishment of a functional blood vessel network), after implantation of the cell-type compared to a corresponding naturally-occurring control tissue. In another preferred embodiment, the tissue or organ has at least a 5%, 10%, 20%, 30%, 40%, 50%, 60%, or even 70%, 80%, 90% or 95% increase in cell number compared to a corresponding control tissue or organ. In another embodiment, the tissue or organ further comprises cells derived from the group consisting of bladder, brain, nervous tissue, glial tissue, esophagus, fallopian tube, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, and cartilage. In another embodiment, the tissue or organ has at least a 5%, 10%, 20%, 30%, 40%, 50%, 70%, 80%, 90%, 100%, 150%, 200%, or even a 300%, 400%, or 500%, increase in biological function compared to a corresponding, naturally-occurring tissue or organ. In one embodiment, the biological function of the tissue or organ is digestion, excretion of waste, secretion, electrical activity, muscle activity, hormone production, or other metabolic activity. Methods for assaying the biological function of virtually any organ are routine, and are known to the skilled artisan (e.g., Guyton et al., Textbook of Medical Physiology, Tenth edition, W.B. Saunders Co., 2000).

In another aspect, the invention features an organ that includes a tissue of the invention. Optionally, the organ is an engineered organ comprising a microvascular scaffold. In various embodiments, the organ is a bladder, brain, nervous tissue, glial tissue, esophagus, fallopian tube, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, or cartilage. In preferred embodiments, the tissue includes one or more cell-types derived from bladder, brain, nervous tissue, glial tissue, esophagus, fallopian tube, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, or cartilage. Optionally, blood vessel formation, biological function, or cell number of an engineered organ comprising a microvascular scaffold is compared to engineered organs not comprising a microvascular scaffold.

In other embodiments of the preceding aspects, a cell of the invention is transformed with a heterologous nucleic acid. In some embodiments, the administered cells (e.g., adipocytes) are not genetically modified. In other embodiments, the cells are genetically modified to express a bioactive molecule, or heterologous protein or to overexpress an endogenous protein, for example, an angiogenesis-related factor selected from the group consisting of activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin and fibronectin receptor α5β1, Factor X, HB-EGF, HBNF, HGF, HUAF, heart derived inhibitor of vascular cell proliferation, IFN-gamma, IL1, IGF-2 IFN-gamma, integrin receptors, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPARγ, PPARγ ligands, phosphodiesterase, prolactin, prostacyclin, protein S, smooth muscle cell-derived growth factor, smooth muscle cell-derived migration factor, sphingosine-1-phosphate-1 (S1P1), Syk, SLP76, tachykinins, TGF-beta, Tie 1, Tie2, TGF-β, and TGF-β receptors, TIMPs, TNF-alpha, TNF-beta, transferrin, thrombospondin, urokinase, VEGF-A, VEGF-B, VEGF-C, VEGF-D, VEGF-E, VEGF, VEGF₁₆₄, VEGI, EG-VEGF, VEGF receptors, PF4, 16 kDa fragment of prolactin, prostaglandins E1 and E2, steroids, heparin, 1-butyryl glycerol (monobutyrin), or nicotinic amide.

In preferred embodiments of any of the preceding aspects, the cells administered include, but are not limited to, perivascular cell (e.g., pericytes), vascular smooth muscle cells, mesenchymal precursor cells (e.g., embryonic stem cells or 10T1/2 cells), mesenchymal cells, preadipocytes (e.g., TA1, 3T3-L1, 3T3-F442A, or Ob17), adipocytes, murine embryonic fibroblasts, fibroblast cell lines (e.g., NIH 3T3, Swiss 3T3, BalbC 3T3), and tumor activated stromal cells (e.g., GFP-positive cells isolated from tumors grown in VEGF-GFP mice, EF1a-GFP mice, or Tie2-GFP mice).

In other preferred embodiments of any of the preceding aspects, blood vascular endothelial cells or lymph vascular endothelial cells are derived from freshly prepared primary culture endothelial cells (obtained from the patient who is to receive the graft or from a donor), stem cells that differentiate into endothelial cells, or endothelial cells generated from stem cells in vitro. Sources of embryonic stem cells include, but are not limited to, bone marrow derived stem cells or cord blood derived cells. Additional sources include HUVEC, lymphatic endothelial cells, embryonic stem cells, and endothelial pregenitor cells .

In other preferred embodiments of any of the preceding aspects, the matrix may include a collagen gel, a polyvinyl alcohol sponge, a poly(D,L-lactide-co-glycolide) fiber matrix, a polyglactin fiber, a calcium alginate gel, a polyglycolic acid mesh, polyester (e.g., poly-(L-lactic acid) or a polyanhydride), a polysaccharide (e.g. alginate), polyphosphazene, or polyacrylate, or a polyethylene oxide-polypropylene glycol block copolymer. Matrices may be produced from proteins (e.g. extracellular matrix proteins such as fibrin, collagen, and fibronectin), polymers (e.g., polyvinylpyrrolidone), or hyaluronic acid. Synthetic polymers may also be used, including bioerodible polymers (e.g., poly(lactide), poly(glycolic acid), poly(lactide-co-glycolide), poly(caprolactone), polycarbonates, polyamides, polyanhydrides, polyamino acids, polyortho esters, polyacetals, polycyanoacrylates), degradable polyurethanes, non-erodible polymers (e.g., polyacrylates, ethylene-vinyl acetate polymers and other acyl substituted cellulose acetates and derivatives thereof), non-erodible polyurethanes, polystyrenes, polyvinyl chloride, polyvinyl fluoride, poly(vinylimidazole), chlorosulphonated polyolifins, polyethylene oxide, polyvinyl alcohol, teflon.RTM., and nylon.

The matrix of any of the preceding aspects may be cultured with any cell of the invention, or may be administered prior to, during, or after the implantation of any cell of the invention. This administration may be by any method known to the skilled artisan (e.g., injection or surgical implantation).

In some embodiments of any of the above aspects, the cells of the invention are derived from a mammalian donor (e.g., pig or primate) of a different species than the recipient (e.g., human).

In another aspect the invention features a method of identifying a compound that modulates blood vessel formation. This method includes the steps of (a) culturing a first cell selected from a first cell selected from the group consisting of blood vascular endothelial cells, lymph vascular endothelial cells, or endothelial cell lines; with a second cell selected from the group consisting of preadipocytes, adipocytes, perivascular cells, vascular smooth muscle cells, mesenchymal precursor cells, mesenchymal cells, and fibroblasts under conditions that allow blood vessel formation; (b) contacting the culture of step (a) with a test compound; (c) measuring the blood vessel formation in the culture; and (d) determining whether the test compound modulates blood vessel formation in the culture relative to a control culture not contacted with the test compound. Desirably, the first and second cells are cultured in the presence of a matrix or a tissue or an organ.

In preferred embodiments, the test compound is part of a test mixture such as a cell lysate, a lysate from a tissue, or a library. A compound that modulates blood vessel formation can either increase or decrease blood vessel formation (e.g., angiogenesis, vasculogenesis, formation of an immature blood vessel network, blood vessel remodeling, blood vessel stabilization, blood vessel maturation, blood vessel differentiation, or establishment of a functional blood vessel network) in the culture, matrix, tissue or organ by at least 5%, 10%, 20%, 25%, 30%, 40%, or 50%, 60%, 70%, 80%, 90%, or even by as much as 100%, 150%, or 200% compared to a corresponding, blood vessel not contacted with the compound.

By “blood vessel formation,” “blood vessel engineering,” or “engineering blood vessels” is meant the dynamic process that includes one or more steps of blood vessel development and/or maturation, such as angiogenesis, vasculogenesis, formation of an immature blood vessel network, blood vessel remodeling, blood vessel stabilization, blood vessel maturation, blood vessel differentiation, or establishment of a functional blood vessel network.

Methods for measuring blood vessel formation and maturation are standard in the art and are described, for example, in Jain et al., (Nat. Rev. Cancer 2:266-276, 2002). During early blood vessel formation, immature vessels resemble the vascular plexus during development, by having relatively large diameters and lacking morphological vessel differentiation. Over time, the mesh-like pattern of immature angiogenic vessels gradually mature into functional microcirculatory units, which develop into a dense capillary network having differentiated arterioles and venules.

The number of blood vessel segments and total length of blood vessels per unit tissue area increases, accompanied by a decrease in mean vessel diameter as the blood vessels are remodeled. The total volume of blood vessels per unit tissue area typically does not change during the remodeling process. The blood vessel size distribution typically narrows with the remodeling of the vessel network.

By “angiogenesis” is meant the growth of new blood vessels originating from existing blood vessels. Methods for measuring angiogenesis are standard, and are described, for example, in Jain et al. (Nat. Rev. Cancer 2:266-276, 2002). Angiogenesis can be assayed by measuring the number of non-branching blood vessel segments (number of segments per unit area), the functional vascular density (total length of perfused blood vessel per unit area), the vessel diameter, or the vessel volume density (total of calculated blood vessel volume based on length and diameter of each segment per unit area).

By “vasculogenesis” is meant the development of new blood vessels originating from stem cells, angioblasts, or other precursor cells. These stem cells can be recruited from bone marrow endogenously or implanted therapeutically.

By “blood vessel maturation” is meant the structural remodeling and/or differentiation of an immature blood vessel network. In some embodiments, blood vessel maturation includes the elimination of extraneous vessels. In other embodiments, blood vessel maturation includes forming a network of blood vessels of different sizes and wall structures (e.g., capillaries, venules, veins, arterioles, and/or arteries). In some embodiments, a mature functional blood vessel network includes some vessels having at least two or more layers, including an endothelial cell layer, a basement membrane, and a perivascular cell layer. In some embodiments, a mature functional blood vessel network includes small arteries and arterioles, which decrease their size with branching, typically 15-40 μm in diameter, terminal arterioles, immediate upstream of capillaries, which typically range in size from 10-15 μm in diameter, capillaries, which typically range in size from 5-10 μm in diameter, post-capillary venules, which typically range in size from 10-20 μm, collecting venules, which typically range in size from 15-25 μm, and venules and small veins which increase their size with gathering typically 20-50 μm in diameter. Optionally, the functional blood vessel network is integrated into a larger circulatory system that includes large veins and arteries.

By “microvascular scaffold” is meant a network of blood vessels. In one preferred embodiment, a microvascular scaffold is an isolated immature network of capillaries, not yet perfused, that persists for at least 24 or 48 hours. More preferably, a microvascular scaffold is a perfused network of functional blood vessels capable of supplying oxygen and nutrients to a tissue or organ and carrying away waste products. In another preferred embodiment, a microvascular scaffold is a mature network of stable differentiated blood vessels that comprises arterioles, venules, and/or other blood vessels that is integrated into the circulatory system of a tissue, organ, or mammal.

By “matrix” is meant the substance that fills the spaces between isolated cells in culture. For some applications, a matrix is an adhesive substrate used to coat a glass or plastic surface prior to cell culture. For some applications, cells are embedded in a matrix, or injected into a matrix already implanted at a desired site. For other applications, a matrix provides a physical support and an adhesive substrate for isolated cells during in vitro culturing and subsequent in vivo implantation. The matrix configuration is dependent on the tissue that is to be treated, repaired, or produced, but desirably, the matrix is a pliable, biocompatible, porous template that allows for vascular growth.

By “modulates” is meant causing an increase or a decrease in blood vessel formation as measured by standard assays such as those described above. Desirably, a compound that modulates blood vessel formation will increase or decrease blood vessel formation (e.g., angiogenesis, vasculogenesis, formation of an immature blood vessel network, blood vessel remodeling, blood vessel stabilization, blood vessel maturation, blood vessel differentiation, or establishment of a functional blood vessel network) in a tissue or organ or microvascular scaffold by at least 5%, 10%, 20%, 25%, 30%, 40%, or 50%, 60%, 70%, 80%, 90%, or even by as much as 100%, 150%, or 200% compared to a control not treated with the compound.

By “mammal” is meant any warm-blooded animal including but not limited to a human, cow, horse, pig, sheep, goat, bird, mouse, rat, dog, cat, monkey, baboon, or the like. It is most preferred that the mammal be a human.

By “organ” is meant a collection of cells that perform a biological function. In one embodiment, an organ includes, but is not limited to, bladder, brain, nervous tissue, glial tissue, esophagus, fallopian tube, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, and cartilage. The biological function of an organ can be assayed using standard methods known to the skilled artisan.

By “mesenchymal cell” is meant a cell typically derived from the mesodermal layer which are pluripotent in the embryonic body and can develop into any of the types of connective or supporting tissues, smooth muscle, vascular endothelium, or blood cells.

By “mural cell” is meant a nonendothelial cell enclosed within the basement membrane of a blood vessel.

By “parenchymal cell” is meant a cell that constitutes the essential part of an organ as distinguished from associated connective tissue, blood vessels, and supporting cells.

By “perfused” is meant filled with flowing blood.

By “tissue” is meant a collection of cells having a similar morphology and function.

By “tissue or organ in need of increased blood vessel formation or engineered blood vessels” is meant any organ or tissue that is impaired as a result of damage or disease, which can result in the loss, atrophy, dysfunction, or death of cells in the tissue or organ. Also included in this definition is any transplanted tissue or organ and any synthetic or engineered tissues or organs.

By “deficiency of a particular cell-type” is meant fewer of a specific set of cells than are normally present in a tissue or organ not having a deficiency. For example, a deficiency is a 5%, 10%, 15%, 20%, 30%, 40%, 50%, 60%, 70%, 80%, 90%, or even 100% deficit in the number of cells of a particular cell-type (e.g., parenchymal cells, preadipocytes, adipocytes not having a genetic modification, perivascular cells, vascular smooth muscle cells, mesenchymal precursor cells, mesenchymal cells, endothelial cells, endothelial precursor cells, or fibroblasts) relative to the number of cells present in a naturally-occurring, corresponding tissue or organ. Methods for assaying cell-number are standard in the art, and are described in (Bonifacino et al., Current Protocols in Cell Biology, Loose-leaf, John Wiley and Sons, Inc., San Francisco, Calif., 1999; Robinson et al., Current Protocols in Cytometry Loose-leaf, John Wiley and Sons, Inc., San Francisco, Calif., October 1997).

By “polypeptide” is meant any chain of amino acids, regardless of length or post-translational modification (for example, glycosylation or phosphorylation).

By “transformed cell” is meant a cell into which (or into an ancestor of which) has been introduced, by means of recombinant DNA techniques, a polynucleotide molecule encoding (as used herein) a polypeptide of the invention.

By “positioned for expression” is meant that the polynucleotide of the invention (e.g., a DNA molecule) is positioned adjacent to a DNA sequence which directs transcription and, for proteins, translation of the sequence (i.e., facilitates the production of, for example, a recombinant polypeptide of the invention, or an RNA molecule).

By “immunological assay” is meant an assay that relies on an immunological reaction, for example, antibody binding to an antigen. Examples of immunological assays include ELISAs, Western blots, immunoprecipitations, and other assays known to the skilled artisan. Immunological assays can be used in standard methods to reassure the function of a tissue or organ. Immunological assays can be used in standard methods to measure the function of a tissue or organ.

The invention provides methods and compositions for repairing diseased or damaged organs, either in vivo, or ex vivo, provides for improved methods of transplantation by enhancing the integration of the transplanted tissue or organ into the host's circulatory system, and provides for improved engineered tissues and organs containing functional microvascular scaffolds. Other features and advantages of the invention will be apparent from the detailed description, and from the claims.

### BRIEF DESCRIPTION OF THE DRAWINGS

FIGS. 1A-1J are images illustrating angiogenesis and vessel remodeling during adipogenesis. FIG. 1A is a picture of a mouse dorsal skin chamber following 3T3-F442A cell implantation. FIG. 1B is a low power microscopic image of FIG. 1A. FIGS. 1C-1F are high power microscopic images of fluorescence contrast enhanced blood vessels at 7 days (FIG. 1C), 14 days (FIG. 1D), 21 days (FIG. 1E), and 28 days (FIG. 1F) after preadipocyte implantation.

FIGS. 1G-1J are graphs of the quantitative analysis of blood vessels during adipogenesis: number of vessel segments in the high power view field (FIG. 1G); vascular length density (FIG. 1H); vessel diameter (FIG. 1I); and calculated blood vessel volume (FIG. 1J) (n=7).

FIGS. 2A-2H are images and graphs illustrating angiogenesis induced by 3T3-F442A preadipocyte cells (FIGS. 2A and 2B) and NIH 3T3 fibroblast cells (FIGS. 2C and 2D). FIGS. 2A-2D are pictures of a mouse dorsal skin chamber following cell implantation. Vessels were visualized at day 14 after implantation by fluorescence microscopy using FITC dextran (2M Dalton). FIGS. 2E-2H are graphs of quantitative analysis of blood vessels: number of vessel segments in the view field (FIG. 2E); vascular length density (FIG. 2F); vessel diameter (FIG. 2G); and calculated blood vessel volume (FIG. 2H). Closed diamonds denote 3T3-F442A preadipocytes (n=7); closed squares denote NIH 3T3 fibroblasts (n=4).

FIGS. 3A-3D are vessel diameter histograms during adipogenesis. Each segment in FIGS. 3A-3D was categorized to a group depending on its diameter and shown as cumulative frequency distribution. FIG. 3A is a vessel diameter histogram at day 7. In this histogram, segment diameters were distributed over a wide range. FIG. 3B and FIG. 3C are vessel diameter histograms at days 14 and 21, respectively. The distribution of vessel diameter shifted leftward and the range became narrower as a result of vessel remodeling with continued adipogenesis. FIG. 3D is a vessel diameter histogram at day 28. In this histogram, most segments (92%) were distributed from 3 to 9 μm in diameter.

FIGS. 4A-4C are pictures of preadipocyte differentiation. FIG. 4A is a transillumination image of differentiated adipocytes in vitro. FIG. 4B is a fluorescence image of differentiated adipocytes in vivo. FIG. 4C is a picture showing the effect of peroxisome proliferator-activated receptor γ (PPARγ) inhibition on adipogenesis. Preadipocytes (1×10⁵ cells) were plated in a 6-well plate and transfected at a multiplicity of infection of 104 plaque forming units/cell using mock- (upper panels) or PPARγ-dominant negative adenovirus (lower panels) and differentiation was promoted by using media containing 10% FBS. Oil Red O (0.3%, 1 hour at room temperature, Sigma, St. Louis, Mo.) staining was performed at day 12 after transfection (magnification, 20×), confirming the inhibition of adipogenesis by the PPARγ-dominant negative construct in murine preadipocytes.

FIG. 5 is a table of angiogenic gene array analysis performed using Mouse Angiogenesis GEArray Q Series (Superarray Inc., Bethesda, Md.), which contains 96 genes known to be involved in angiogenesis, according to the manufacturer's instructions. “(+)” denotes detectable; in this case, intensity was higher than background, and hybridization was confirmed by visual inspection. “N.D.” denotes not detectable; in this case, intensity was lower or close to background. Samples are from cultured 3T3-442A cells. Preadipocytes were cultured in maintenance media (10% FCS). “PPAR-DN” denotes PPARγ dominant negative mutant receptor transduced cells cultured in maintenance media. Adipocytes were cultured in differentiation media (10% FBS). More than 2-fold difference in normalized intensity compared to preadipocytes is noted.

FIGS. 6A-6H are images and graphs illustrating the effect of PPARγ inhibition on angiogenesis. FIGS. 6A-6D are fluorescence images of blood vessels at 21 days (FIGS. 6A and 6C), and 28 days (FIGS. 6B and 6D) after mock- (FIGS. 6A and 6B) and PPARγ dominant negative- (FIGS. 6C and 6D) transfected preadipocytes implantation. FIGS. 6E-6H are graphs of the quantitative analysis of blood vessels: number of vessel segments in the high power view field (FIG. 6E); vascular length density (FIG. 6F); vessel diameter (FIG. 6G); and calculated blood vessel volume (FIG. 6H). There was no difference between two different control cells, mock-transfected preadipocytes (n=3) and EF1a-GFP 3T3-F442A cells (n=3). Thus, these two groups are combined as control for data presentation and statistical analysis. Filled circle, control (n=6); open square, PPARγ dominant negative-transfected preadipocytes (n=5). *P<0.01 as compared with corresponding control by two-tailed t-test.

FIG. 7 is a picture of VEGF mRNA expression during adipocyte differentiation. Differentiation was initiated when the cell became confluent by addition of differentiation media. For control, the cells were cultured in the maintenance media without insulin. At day 8 and 12 after replacement of the media, total cellular RNA was obtained from 3T3-F442A adipocytes, and 10 μg aliquots were electrophoresed, blotted, and hybridized to the VEGF cDNA, 18S and 28S as described herein.

FIG. 8 is a table of PCR primers (SEQ ID NOs: 1-25). Northern blots were probed with PCR-generated cDNA fragments. Nested primers were used to generate specific amplification products. Primers for PCR were synthesized based on Ang1, Ang2, and aP2 mouse sequences (GenBank accession numbers AAB50558, NM_{—}007426 and NM_{—}024406). Primers for RT-PCR were synthesized based on the GenBank sequence information. These primers were designed to amplify fragments of about 300 basepairs. Twenty-five cycles each of 20 seconds at 93° C., 20 seconds at 55° C., and 30 seconds at 72° C. were performed. PCR products were resolved by electrophoresis on a 2% agarose gel. The gel was stained with ethidium bromide, and bands were visualized on an UV transilluminator.

FIG. 9A is an image illustrating the effect of VEGF on preadipocyte differentiation and proliferation. To investigate the effects of VEGF on the in vitro differentiation of preadipocytes, 3T3-F442A cells were grown to confluence in media supplemented with calf serum (FCS, maintenance media), and exposed to increasing concentrations of murine recombinant VEGF₁₆₄ (R&D Systems, Minneapolis, MN) from 0-100 ng/ml. Mouse recombinant VEGF₁₆₄ did not induce differentiation in preadipocytes cultured in 10% FCS (maintenance media), and did not increase the differentiation rate in cells treated with 10% FBS (differentiation media).

FIG. 9B is a graph depicting the results of proliferation assays in the presence or absence of VEGF₁₆₄. 500 preadipocytes and fibroblasts were plated in 96-well plates, and mouse recombinant VEGF₁₆₄ (50 ng/ml) and PBS were added. An MTT assay was performed at day four, when the cells were still subconfluent in all wells. Culture media were changed with 100 μl of fresh media, and 10 μl of sterile tetrazolium salt, MTT (3[4,5-dimethylthiazol-2-yl]-2,5-diphenyl-tetrazolium bromide, Sigma) was added in each well and incubated for four hours at 37° C. Finally, 100 μl of 10% SDS were added, and after incubation at 37° C. overnight, the plate was read at 490 nm. The optical density values were normalized to that of the PBS treated cells and used as a measure of viability.

FIG. 10 is a photomicrograph showing engineered microvessel growth detected by multiphoton laser scanning microscopy 55 days after implantation of human umbilical vein endothelial cells (HUVECs) and 3T3-F442a co-cultured cells. HUVECs are shown in green (EGFP labeled), and functional blood vessels, visualized by injected rhodamine dextran, are shown in red.

FIG. 11 is a series of images showing engineered blood vessels from 12 hours to 11 months.

FIG. 12 is an image showing three-dimensional engineered vessels from HUVECs and 10T1/2 cells or HUVECs alone seeded in three-dimensional constructs and implanted in mice. (HUVECs, green; functional blood vessels, red).

FIG. 13 is a combination of graphs showing temporal changes in the density of vessels engineered from HUVECs and 10T1/2 cells or HUVECs alone seeded in three-dimensional constructs and implanted in mice.

FIG. 14 is an image showing the incorporation of 10T1/2 cells (expressing EGFP, green) into perfused vessel walls (red).

FIG. 15 is a combination of images showing the vasoconstriction of engineered vessels after local administration of 100 nM endothelin-1 (ET-1) on the surface of the tissue construct forty-four days after EGFP-HUVEC and 10T1/2 co-implantation using intravital microscopy. Scale bars: 100 μm.

FIGS. 16A and 16B are graphs showing the perfusion of engineered vessels over time. FIG. 16A is a graph showing the fraction of perfused engineered vessels in the upper, middle and lower layer of the construct separately using projected images of 20 μm thickness in each layer. Blood perfusion was initiated in the lower layer, then in the middle and upper layers (mean±SEM: n=4: asterisk and dagger denote P<0.05 between lower and upper or middle and upper layers, respectively). FIG. 16B is a graph showing the long-term follow up of vascular densities of perfused and non-perfused engineered vessels.

FIGS. 17A-17G are images showing the histological analyses of engineered vessels. FIG. 17A is an image showing cross-sectional images including both implanted engineered construct (upper parts) and host tissue (lower parts) by double staining of human (green) and mouse (red) CD31. Most vessels in the 3-D construct were positive for human CD31, confirming their HUVEC origin. White arrows indicate the occasional host-derived endothelial cell. Large host vessels were observed outside the gel. (Day 84; scale bar: 50 μm). FIG. 17B is an image showing cross-sectional images including both implanted engineered construct (upper parts) and host tissue (lower parts) by immunohistochemistry for α-smooth muscle actin (α-SMA, red) in the tissue construct 35 days after the implantation. The orange staining indicates vessel fortification by α-SMA positive 10T1/2 cells. Vessels in underlying host tissue are also α-SMA positive (lower left). Scale bar: 50 μm. FIGS. 17C and 17D are images showing double staining of human CD31 (green) and α-SMA (red) of the engineered construct. Both large and small engineered vessels were covered with α-SMA positive cells. (Day 35; scale bar: 50 μm). FIG. 17E is an image showing double staining of GFP (green) and α-SMA (red) in the EGFP-10T1/2 and HUVEC construct. A large vessel is covered by α-SMA positive cells which partially co-localized with GFP. This vessel wall is probably composed of both implanted 10T1/2 cells and host cells. (Day 35; scale bar: 50 μm). FIG. 17F is an image showing α-SMA staining of the engineered vessels using the immunoperoxidase method. This engineered vessel was derived from a HUVEC-alone gel. (Day 137; scale bar: 50 μm). FIG. 17G is an image showing hematoxylin and eosin staining of the engineered tissue construct. Arterioles have thicker vessel wall with circumferential mural cells (black arrow) and venules have a thin layer of mural cells (arrow head). (Day 56; scale bar: 100 μm).

### DESCRIPTION OF THE INVENTION

The present invention features improved methods and compositions for tissue and organ transplantation, the repair of diseased or damaged tissues and organs, and replacement tissue and organ engineering.

As reported in more detail below, human umbilical vein endothelial cells embedded in three-dimensional matrices formed functional vessels in vivo, and the engineered vessels were stabilized by co-implantation with mesenchymal precursor cells (10T1/2) or by preadipocytes (3T3-F442A). In addition, preadipocytes or differentiated adipocytes enhanced and recruited host vessels to infiltrate the three-dimensional matrices. The discovery that preadipocytes induced vigorous angiogenesis in vivo, and that the newly formed vessels subsequently remodeled into a mature network consisting of arterioles, capillaries, and venules provides for improved therapeutic methods of tissue and organ transplantation, methods for stabilizing and repairing damaged tissues or organs, and improved methods for the engineering of tissues and organs.

For successful tissue and organ transplantation, the grafted tissue or organ must be rapidly integrated into the host's circulatory system. Applicants' discovery that preadipocytes and mesenchymal precursor cells induced not only angiogenesis but also subsequent vessel remodeling and maturation resulting in the creation of long-lasting vascular networks provides for improved methods of tissue and organ transplantation. Administering preadipocytes or mesenchymal precursor cells to grafted tissue or organs induces blood vessel formation and a functional blood vessel network, and enhances integration of the graft into the recipient's circulatory system.

Many patients remain on transplantation waiting lists for months or even years. Thus, a need exists for therapeutic methods that would stabilize a patient's damaged organ while the patient waits for transplantation or repair a diseased or damaged organ. The discovery that administering preadipocytes or mesenchymal cells induces blood vessel formation and a functional blood vessel network provides for the stabilization or repair of damaged or diseased tissues and organs, thereby improving tissue or organ function. Such methods may prolong organ life while a patient waits for a donor organ, or may obviate the need for transplantation.

In addition, shortages in organ donation could be offset if transplantable tissues or organs could be engineered. Successful tissue and organ engineering has been limited by failures in engineering functional blood vessels. The discovery that endothelial cells formed functional microvascular scaffolds when cultured with preadipocytes or mesenchymal precursor cells provides for the culture of microvascular scaffolds whose interstices are filled with parenchymal cells from virtually any target-tissue thus allowing the engineering of replacement tissues and organs.

In addition, the adipogenesis-organogenesis and three-dimensional models of blood vessel formation discovered by the applicants can also be used to screen or compounds that can promote or prevent blood vessel formation. These compounds can then be used as novel therapeutics to induce or to block angiogenesis as needed. These models can also be used to address the mechanisms of normalization and maturation of blood vessels, and to develop and test novel strategies for tissue engineering, organogenesis, and therapeutic blood vessel formation and blood vessel engineering.

These methods are described in detail below.

Mouse Model of Angiogenesis

Methods for producing an engineered microvascular scaffold were developed using a mouse model of angiogenesis. This model was developed using an established preadipocyte cell line (3T3-F442A) that is able to differentiate into mature adipocytes in culture (Gregoire et al., Physiol. Rev. 78:783-809, 1998). 3T3-F442A cells give rise to vascularized fat pads in immunodeficient mice (Mandrup et al., Proc. Natl. Acad. Sci. U.S.A. 94:4300-4305, 1997). 3T3-F442A preadipocytes (a generous gift from Dr. Bruce Spiegelman, Dana-Farber Cancer Institute, Boston, Ma.) and their parental cell line (NIH 3T3 fibroblasts) were maintained in Dulbecco's Minimum Essential Medium (DMEM, Gibco BRL, Grand Islands, N.Y.), supplemented with 10% calf serum, glucose, L-glutamine, penicillin, and streptomycin. For cell identification in vivo, preadipocytes were transfected by the calcium phosphate method with GFP under the EF1a promoter; these cells are referred to as GFP/3T3-F442A.

These preadipocytes were implanted in the dorsal skin-fold chamber (Jain et al., Nat. Rev. Cancer 2:266-276, 2002) of male, 8-12-weeks-old severe combined immunodeficient (SCID) mice to monitor angiogenesis during fat formation. In particular, dense cell pellets containing 2×10⁵ mouse preadipocytes (or NIH 3T3 fibroblasts as a control) were implanted in the center of the dorsal skinfold chamber. These mice were bred and maintained in a defined flora facility. In vivo microscopy was performed 1-2 times a week for as many as four weeks after the implantation. Implants were then analysed for vascular parameters as described previously (Jain et al., supra).

For each animal, the implant was analyzed at five randomly chosen locations per time point. The number of non-branching blood vessel segments (number of segments per unit area), the functional vascular density (total length of perfused blood vessel per unit area), the vessel diameter, and the vessel volume density (total of calculated blood vessel volume based on length and diameter of each segment per unit area) were determined as described elsewhere (Jain et al., supra). Angiogenesis and subsequent vessel remodeling were analyzed following the implantation of NIH 3T3, 3T3-F442A, GFP/3T3-F442A, or GFP/3T3-F442A cells infected with a recombinant adenovirus encoding a PPARγ dominant negative mutant receptor or mock adenovirus (Gurnell et al., J. Biol. Chem. 275:5754-5759, 2000).

Characterization of Angiogenesis in Mouse

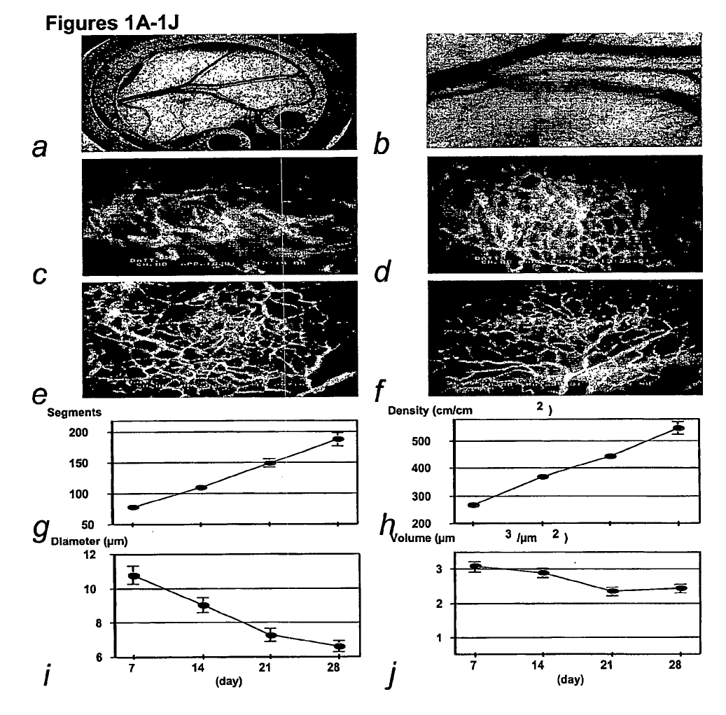

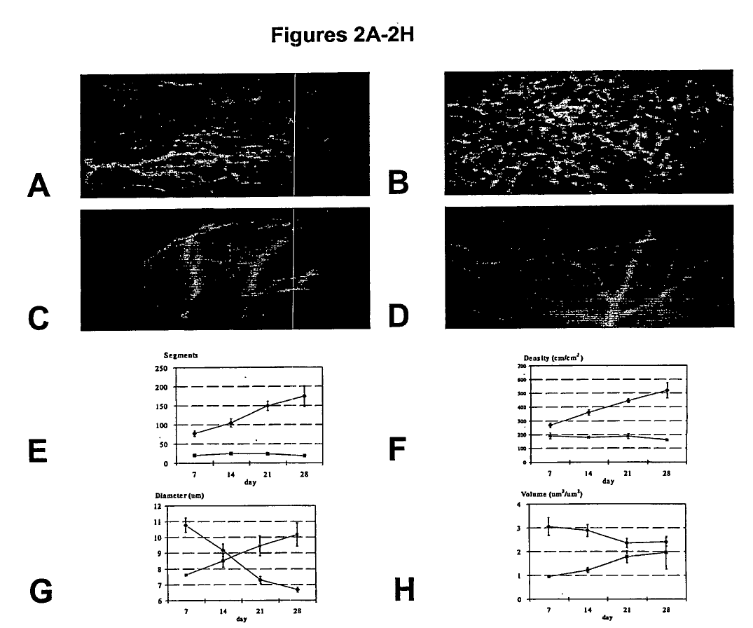

Mice with transplanted preadipocytes displayed reddened tissue at sites of active angiogenesis (FIG. 1A). Angiogenic vessels were detected in implanted 3T3-F442 cell pellets located on top of host subcutaneous tissues and striated muscle which contained pre-existing host vessels (FIG. 1B). The angiogenic vessels were specifically induced by the 3T3-F442A preadipocytes since control fibroblasts (NIH 3T3 cells) failed to induce detectable vessel formation (FIGS. 2A-2H).

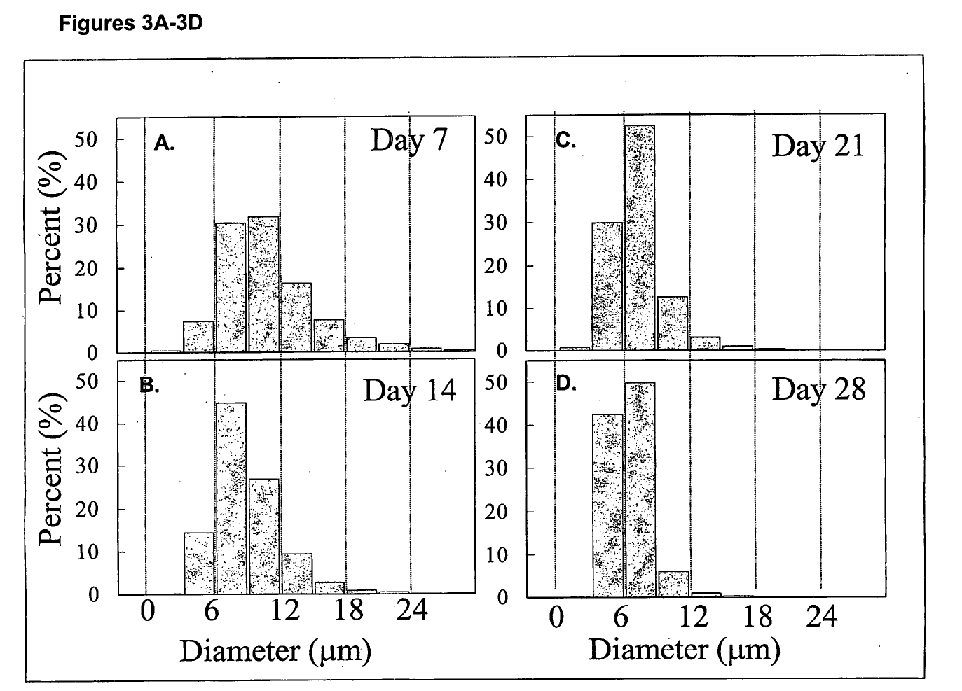

New vessels induced by the preadipocyte implant appeared immature, resembling the vascular plexus during development, having relatively large diameters and lacking morphological vessel differentiation (FIG. 1C). Over time, the vessel network induced by the preadipocyte implant gradually matured (FIGS. 1C-1F). Mesh-like patterns of angiogenic vessels developed into a dense capillary network (FIGS. 1C-1E), with differentiated arterioles and venules (FIG. 1F). The number of blood vessel segments (FIG. 1G) and total length of blood vessels per unit tissue area (FIG. 1H) increased, accompanied by a decrease in mean vessel diameter (FIG. 1I) as the blood vessels remodeled. The total volume of blood vessels per unit tissue area did not change during the remodeling process (FIG. 1J). The blood vessel size distribution narrowed with the remodeling of the vessel network (FIGS. 3A-3D). These findings indicate that preadipocytes induce angiogenesis in vivo and that these vessels are remodeled into an efficient network with mature vessel architecture characterized by small diffusion distance from vessels to parenchymal cells.

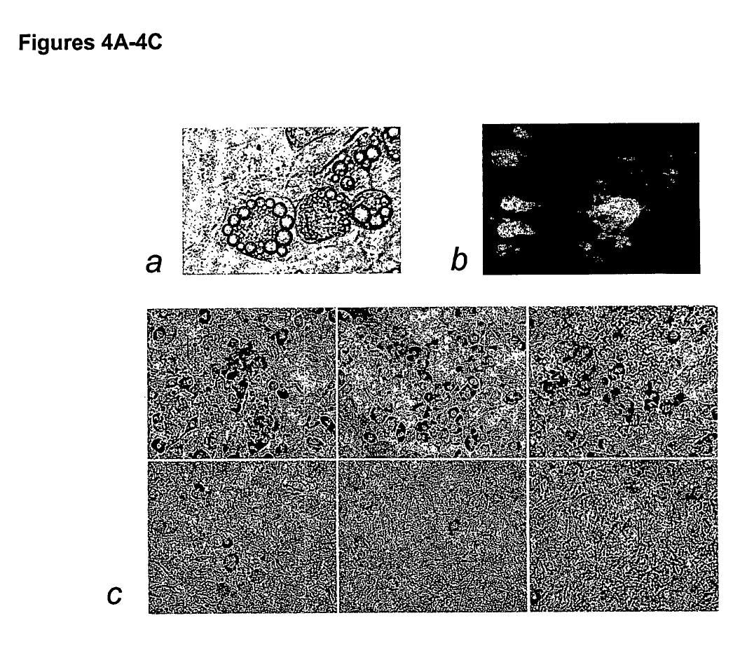

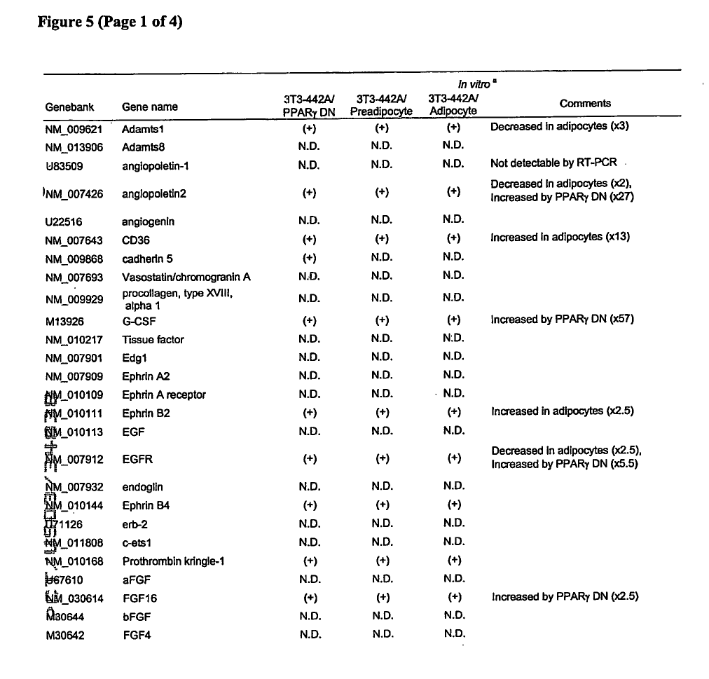

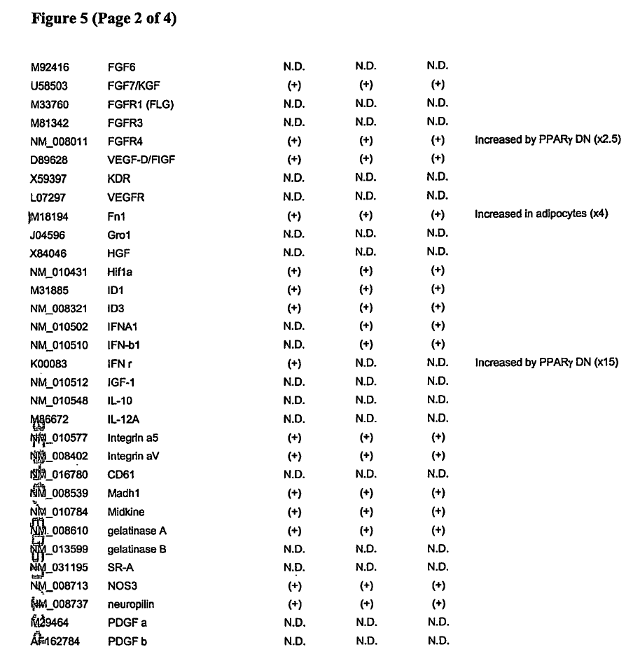

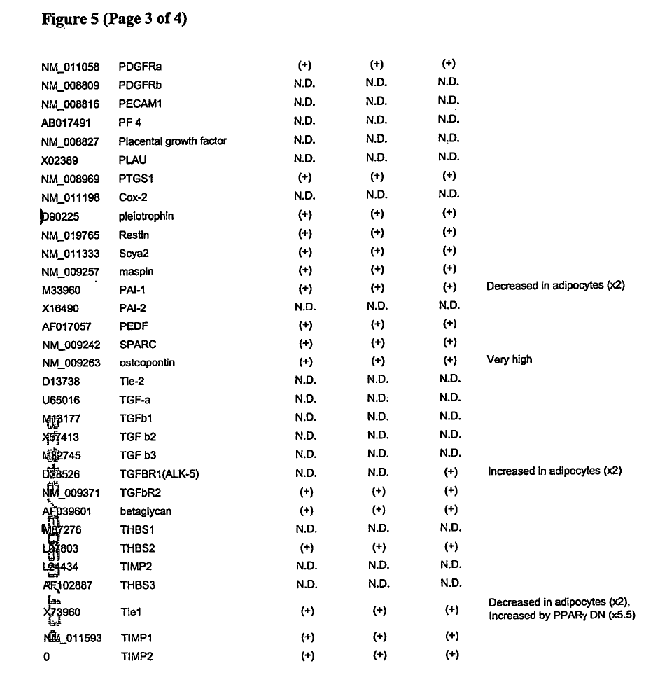

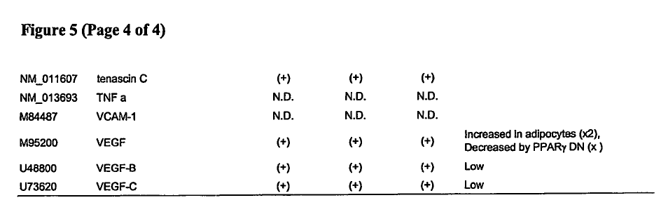

To clearly distinguish between implanted cells and host-derived cells in vivo, 3T3-F442A cells were detected by constitutively expressing the green fluorescent protein (GFP) gene under the control of the EF1α promoter. Cytoplasmic GFP fluorescence allowed the detection of the implanted cells in vivo. The implanted preadipocytes began to differentiate into adipocytes several days after implantation and most of the cells acquired a mature phenotype after four weeks. Differentiation into adipocytes was accompanied by the accumulation of triglyceride-containing vesicles in the cell cytosol (FIG. 4A), which exhibited a granular fluorescence (FIG. 4B). Most of the cells acquired a mature phenotype after four weeks, and adipocyte differentiation was confirmed by the expression of the adipocyte-specific genes aP2 and CD36 (FIG. 5), (Bernlohr et al., Biochem. Biophys. Res. Commun. 132:850-855, 1985; Spiegelman et al., J. Biol. Chem. 258:10083-10089, 1983; and Abumrad et al., J. Biol. Chem. 268:17665-17668, 1993).

Requirement of PPARγ for Adipogenesis

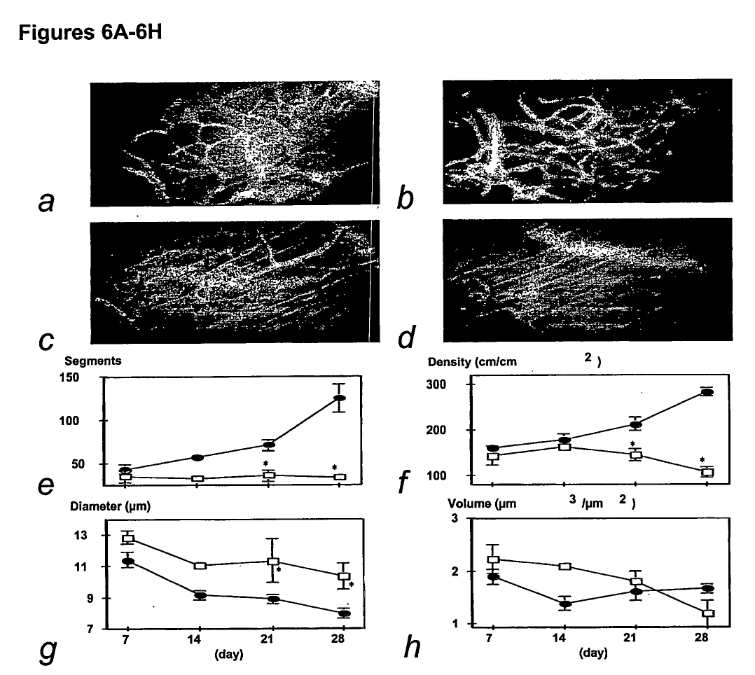

Activation of peroxisome proliferator-activated receptor γ (PPARγ) is required for adipocyte differentiation (Lazar, Genes & Dev. 16:1-5, 2002 and Willson et al., Annu. Rev. Biochem. 70:341-367, 2001). To characterize the link between preadipocyte differentiation and angiogenesis, a dominant negative PPARγ mutant construct was introduced into 3T3-F442A cells prior to implantation using an adenoviral vector, as described previously (Gurnell et al., J. Biol. Chem. 275:5754-5759, 2000). Expression of the mutant receptor prevented the differentiation of 3T3-F442A cells in vitro (FIG. 4B). Control mock-transfected preadipocytes formed fat tissue and induced extensive angiogenesis when implanted in vivo (FIGS. 6A, 6B, and 2E-2H), while cells expressing the PPARγ dominant negative mutant receptor formed no fat tissue and induced reduced angiogenesis when implanted in vivo. Underlying host blood vessels remained visible for the duration of the experiment (FIGS. 6C and 6D) when PPARγ dominant negative cells were implanted. These dominant negative cells remained undifferentiated and expressed lower messenger RNA levels of aP2. Thus, the activation of PPARγ is required for adipogenesis and subsequent angiogenesis in vivo.

Expression of VEGF and Other Angiogenesis Related Factors in Adipose Tissue

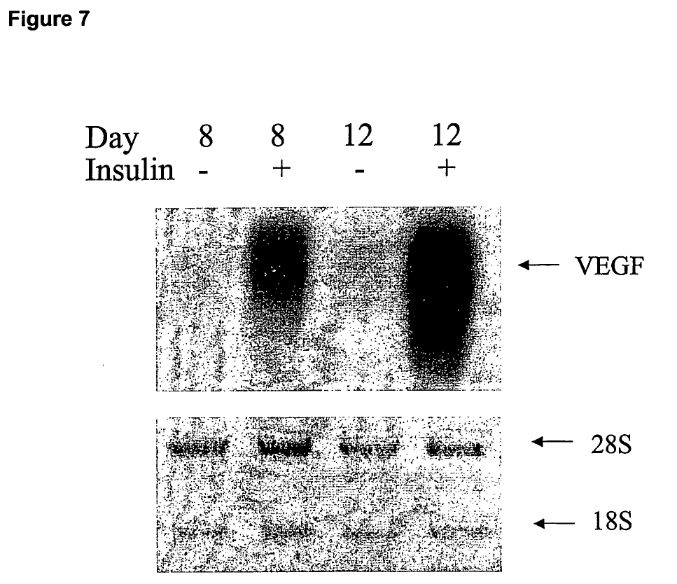

Angiogenesis often precedes adipose tissue formation in developing tissue. VEGF is the most potent and critical angiogenic factor in both physiological and pathological angiogenesis. VEGF is highly expressed in adipose tissue and its expression increases during PPARγ ligand- and other stimuli-induced differentiation of preadipocytes into adipocytes (FIGS. 5 and 7) (Zhang et al., J. Surg. Res. 67:147-154, 1997; Claffey et al., J. Biol. Chem. 267:16317-16322, 1992; Soukas et al., J. Biol. Chem. 276:34167-34174, 2001; and Emoto et al., Diabetes 50:1166-1170; 2001). In agreement with these data, expression of VEGF and various other angiogenesis-related genes was found (FIG. 5) in 3T3-F442A cell-derived tissue in vivo.

To analyze expression of angiogenesis-related genes in vivo, 1.5×10⁷ cells suspended in 100 μl of PBS were injected into the flank of SCID mice. For the anti-adipogenesis studies, mice were divided into three groups with the following cell implants: GFP/3T3-F442A, GFP/3T3-F442A expressing PPARγ dominant negative, and GFP/3T3-F442A mock-transfected. For the anti-angiogenesis experiments, GFP/3T3-F442A cells were implanted in three groups of mice. Fat pad formation was allowed to occur for four weeks, the mice were then sacrificed, and the tissue was harvested. The tissue formed by the implanted preadipocytes was recovered using microscissors and fluorescence microscope-guided dissection. Tissue samples were snap-frozen for subsequent RNA extraction.

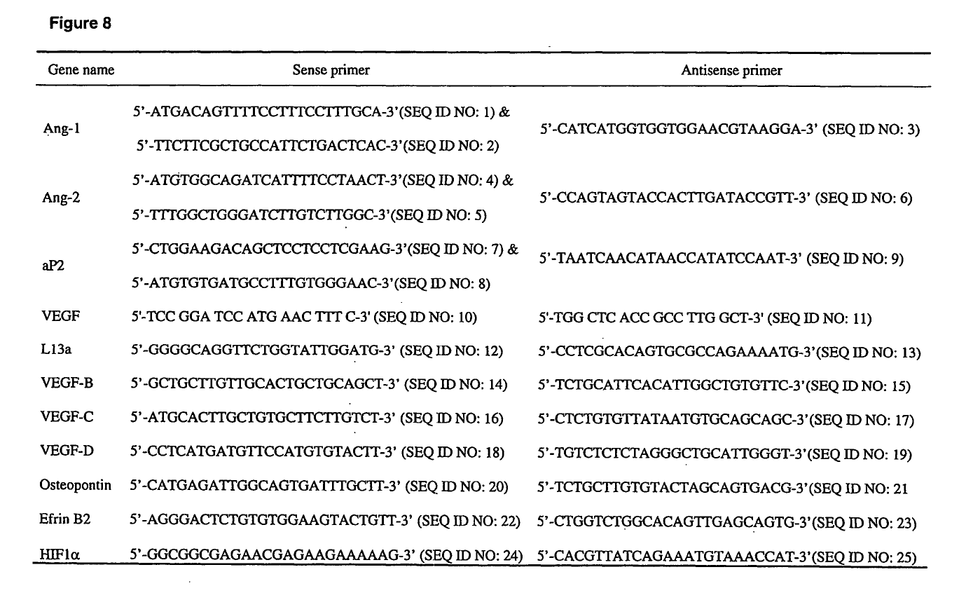

Total RNA was extracted from the cells and the recovered tissue samples using Triazol (Gibco BRL, Grand Islands, N.Y.), following the protocol recommended by the manufacturer. The GFP expression in tissue samples was confirmed by RT-PCR. Primers for RT-PCR and Northern probes are shown in FIG. 8. Ten micrograms of total RNA was separated on a1% agarose/1×MOPS/2% fomaldehyde gel, transferred to nylon membranes in 10×SSC, and UV cross-linked to the membrane. Northern blots were hybridized with random-primed ³²P-labeled probes in QuickHybr Solution (Stratagene, La Jolla Calif.) at 68° C. for one hour. Hybridized blots were washed twice at high stringency in a solution of 0.1×SSC/0.1% sodium dodecylsulfate (SDS) at 55° C. Autoradiography was performed for 1-2 days using a Kodak X-Omat AR film.

Effect of Exogenous VEGF on Adipogenesis In Vitro

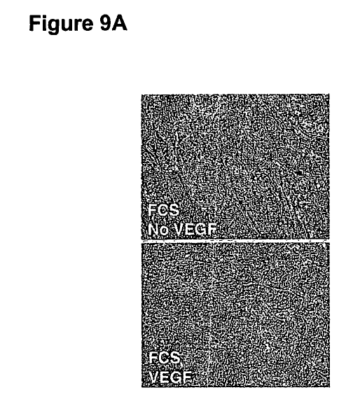

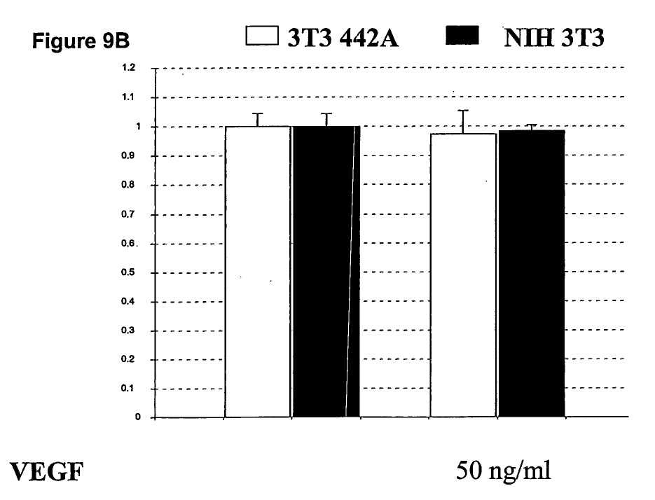

Adipose cell differentiation and proliferation in vitro were not significantly affected by exogenous VEGF, even at doses as high as 100 ng/ml VEGF₁₆₅. The effect of VEGF on adipose cells in culture is shown in FIGS. 9A and 9B. Thus, VEGF signaling may not directly mediate adipogenesis; although, for example, neuropilin-1 was detected in preadipocytes (FIG. 5). Thus, other molecular and microenvironmental changes associated with angiogenesis and/or secondary to VEGF signaling may potentiate adipogenesis in vivo.

Interplay Between Adipose Tissue Formation, Angiogenesis and Vessel Remodeling

These data illustrate the complex interplay between adipose tissue formation, angiogenesis, and vessel remodeling. Angiogenesis was needed for efficient preadipocyte differentiation, but angiogenesis was not triggered without PPARγ activation and subsequent adipocyte differentiation. To analyze the cyclic feedback mechanisms gene array analysis on these tissues was performed at various time points in preadipocytes, adipocytes, and PPARγ dominant negative expressing cells (FIG. 5). Ang-1 expression was increased in 3T3-L1 cells, a preadipocyte cell line, during adipogenesis (Stacker et al., Growth Factors 18:177-191, 2000), but Ang-1 was not detectable in mature adipose tissue (FIG. 5). Ang-2 was expressed in both preadipocytes and adipocytes in vitro and was significantly upregulated in PPARγ-dominant negative expressing cells (FIG. 5). Adipogenesis may be mediated by (i) the auto- and paracrine effects of other angiogenic growth factors on preadipocytes or (ii) the interactions between the matrix associated with angiogenic vessels and preadipocytes (FIG. 5; Varzaneh et al., Metabolism 43:906-912, 1994 and Lilla et al., Am. J. Pathol. 160:1551-1554, 2002).

A salient observation emerging from this study comes from the remodeling and maturation of angiogenic vessels. While aberrant angiogenesis occurs during tumor angiogenesis, driven by excess and/or unbalanced angiogenic factors (Carmeliet et al., Nature 407:249-257, 2000), in this model system new blood vessels mature into a normal network during adipose tissue formation. This is remarkable given that “normal” vasculature is rarely generated in currently available tissue engineering models. Furthermore, these results suggest that the molecular and metabolic microenvironment associated with functional, mature blood vessels potentiates preadipocyte differentiation and adipose tissue formation. Thus, it is likely that the generation of normal microcirculatory units is indispensable for organogenesis. The new adipogenesis-organogenesis model described herein is ideal to address the mechanisms of normalization and maturation of blood vessels, and to develop and test novel strategies for tissue engineering, organogenesis, and therapeutic blood vessel formation and blood vessel engineering.

Angiogenesis in Three-Dimensional Matrices

The ability of preadipocytes to stabilize engineered vessels was analyzed in vivo. Three-dimensional matrices containing co-cultures of implanted human umbilical vein endothelial cells (HUVECs), transfected with EGFP and either 3T3-F442a, preadipocytes, or mesenchymal precursor cells was compared to HUVEC-only implants in a mouse cranial window preparation and in a dorsal skinfold chamber.

Craniotomy and Cranial Window (CW) Preparation

Cranial windows were implanted into mice as previously described (Yuan et al., Cancer Research 54: 4564-4568, 1994). SCID mice (25-30 g) were anesthetized; the head of the mouse was fixed by a stereotactic apparatus, and a longitudinal incision was made between the occiput and forehead. The skin was cut in a circular manner on top of the skull, and the periosteum underneath was scraped off to the temporal crests. A 6-mm circle was drawn over the frontal and parietal regions of the skull bilaterally. Using a high speed air-turbine drill (CH4201S; Champion Dental Products, Placentia, Calif.) with a burr-tip, 0.5 mm in diameter. A groove was made on the margin of the drawn circle, which was then made thinner until the bone flap loosened. Cold saline was applied during the drilling process to avoid thermal injury of the cortical regions. Using a malis dissector, the bone flap was then separated from the dura matter underneath. After removal of bone flap, the gelfoam was placed on the cutting edge and the dura matter was continuosly kept moist with physiological saline. A nick was made close to the sagital sinus. Iris microscissors were passed through the nick. The dura and arachnoid membranes were cut completely from the surface of both hemispheres, and an 8 mm cover glass was glued to the bone with histocompatible cyanoacrylate glue. The mouse was then allowed to recover.

Between seven and ten days after surgery, the cover glass was removed and a piece of gel 3 mm in diameter was put in the center of the window, which was then re-sealed as previously described. Angiogenesis and maturation processes were monitored.

Dorsal Skinfold Chamber (DSC) Preparation

The dorsal skinfold chamber preparation was performed as previously described (Leunig et al., Cancer Research 52: 6553-6560, 1992). SCID mice (25-30 g) were anesthetized. Two symmetrical titanium frames (weight 3.2 g each), which were mirror images of each other (Workshop, Department of Radiation Oncoclogy, MGH) were implanted such that a layer of skin was sandwiched between them. The outer layer of the skin was removed in a 15 mm diameter circle to expose the epidermis, subcutaneous tissue, and striated muscle, which was then covered with a glass coverslip secured into one of the frames by a snap ring. The mouse was allowed to recover from surgery for one day. On the second day after surgery, the mouse was placed in a polycarbonate tube 25 mm in diameter. The snap ring and glass coverslip were removed, and a disk of gel (1 mm in height and 3 mm in diameter) was placed in the chamber. A coverslip was then set into place and secured with a snapring. Angiogenesis and maturation processes were then monitored.

Three-dimensional Cell Constructs

In vitro derived three-dimensional cell constructs of endothelial cells and preadipocytes cultured in a collagen gel were transferred into cranial windows and dorsal skinfold chambers as described above. The three-dimensional cell constructs were produced as follows.

The three dimensional matrix was produced according to the manufacturer's protocol. Type 1 collagen (1.5 mg/ml) was mixed with human plasma fibronectin (90 μg/ml) in 25 mM Hepes and special growth medium for endothelial cells (EGM) at 4° C., and the pH was adjusted to 7.5 using NaOH. Cultured EGFP transduced HUVEC and/or 3T3-F442a (or Swiss 3T3) were trypsinized, and the cells were counted. For gels containing co-cultures of HUVEC and 3T3-F442a (or Swiss 3T3) cells, 0.8×10⁶/ml and 0.2×10⁶/ml collagen solutions, respectively, were used per 1 ml of gel. For control gels ontaining only HUVEC, 1×10⁶ HUVECs were used for 1 ml of gel. The cultured cells were placed into a 15 ml culture tube and spun in a centrifuge to pellet the cells. Then, the supernatant was removed. The cell pellet was then resuspended in 1 ml gel solution, placed in the well of a 12 well plate, and incubated at 37° C. and 5% CO₂ for thirty minutes to allow the gel to polymerize. One ml of endothelial cell growth media modified MCDB131 (EGM) (Cambrex Bio Science Walkerville Inc. Walkersville, Md., CC-3024) media was then added to the well. The cells in the gel were then cultured for twenty-four hours in vitro. The disc-like pieces of gel (3 mm in diameter and 1 mm in height) were transferred to the observation sites (cranial window or dorsal skin fold) approximately twenty-four hours after formation. The cultures containing EGFP transduced HUVEC and 10T1/2 mesenchymal precursor cells were prepared in the same way.

Anastamosis and Microvascular Function

Using intravital and two-photon microscopy, (i) anastomosis between the artificial vessels and the host microvascular system and (ii) microvascular function of the perfused artificial constructs were assessed. Following anaesthesia, intravital microscopy was performed as previously described (Leunig et al., Cancer Research 52: 6553-6560, 1992). Cranial window-bearing mice were fixed on an observation stage to hold the window in position during measurements. Dorsal skinfold mice were positioned in a polycarbonate tube approximately 24 mm in diameter on a polycarbonate stage. The stage was then placed under a microscope. Daily observations were performed using either a 1.25× objective (NA 0.035, Plan Neofluor; Zeiss, Oberkochen, Germany) and a microscope (Axioplan, Zeiss) with a 37-fold magnification on the screen or a 20× long working distance objective (NA 0.4; LD Achroplan) with a final magnification of 570-fold. For general morphology, a transillumination technique (12 V, 100 W halogen lamp, Zeiss), using a green filter for enhancing black/white photomicrography and a conversion filter for converting artificial light of 3200 K into daylight of 5500 K, was used. GFP-expressing endothelial cells were visualized using epifluorescence and fluorescence filters. Observations (intensified CCD camera, C2400-88; Hamamatsu Photonics K.K., Hamamatsu, Japan) were recorded on a videocassette recorder (AG-6500; Panasonic, Secaucus, N.J.) at a rate of 60 frames per second.

Multiphoton Laser-Scanning Microscopy

To determine functionality of engineered vessels, the tail vein was injected with tetramethylrhodamine dextran. Images were taken with multiphoton laser-scanning microscopy (MPLSM). This technology allowed for three-dimensional, high-resolution imaging of endothelial cells at depths extending 400 μm below the surfance of the window. MPLSM was described previously (Brown et al., Nature Medicine 7: 864-868, 2001; Padera et al., Molecular Imaging 1: 9-15, 2002). Briefly, MPLSM consists of a MILLENIAX PUMPED TSUNAMI TI:sapphire laser (Spectra-Physics, Mountain View, Calif.). All data were obtained using a 810-850 nm light. Power at the sample was estimated to be 1-5 mW. The scanhead was a MRC600 with IR optics (Bio-Rad, Hemel Hempstead, England) on a Zeiss AXIOSKOP20 microscope (Zeiss, Jena, Germany) with a LOMO 30×/0.9NA/water (Vermont Optechs, Charlotte, Vermont) and Zeiss 20×/0.5NA/water and 20×/0.4NA/air objective lenses. Non-descanned detection was performed with HC125-02 multiplier (PMT) (Hamamatsu Photonics, Bridgewater, N.J.). Image analysis was performed on SCIONIMAGE software (Scion, Frederick, Md.). After images were taken, the animals were euthanized, and the gel and surrounding tissue were removed for immunohistochemical analysis.

Angiogenesis in Three Dimensional Matrices with Preadipocytes and HUVEC

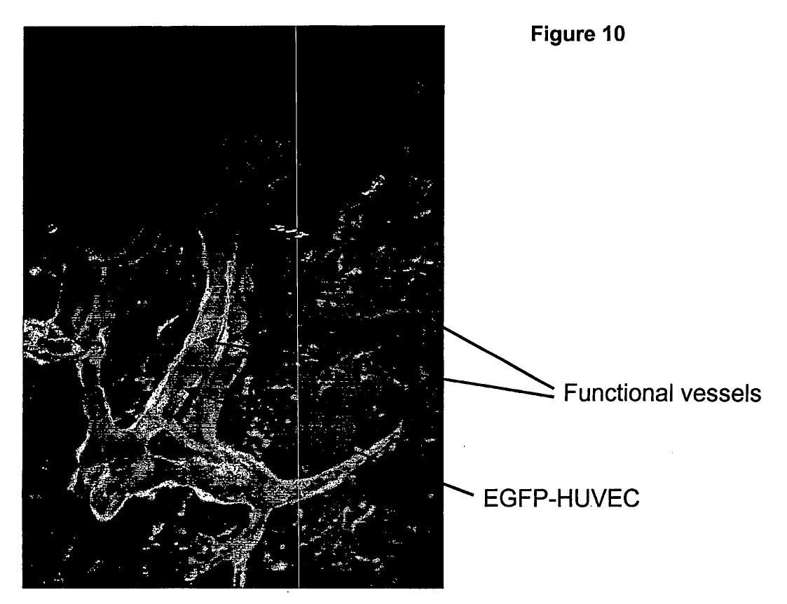

Initially, HUVEC cells co-cultured in collagen matrix with preadipocyte 3T3-F442a displayed a small spindle-shaped morphology. When implanted into an SCID mouse cranial window, HUVECs formed a capillary-like network. At ten days after implantation, blood flow was observed in a small number of engineered vessels (FIG. 10). The number of perfused vessels increased over the course of the experiment. In addition, host vessels were recruited into the implants. These results show that endothelial cells (HUVECs) embedded in three-dimensional matrices are able to form functional vessels in vivo and the engineered vessels are stabilized by co-implantation with adipocyte precursor cells 3T3-F442a.

Angiogenesis in Three-Dimensional Matrices with Mesenchymal Precursor Cells and HUVEC

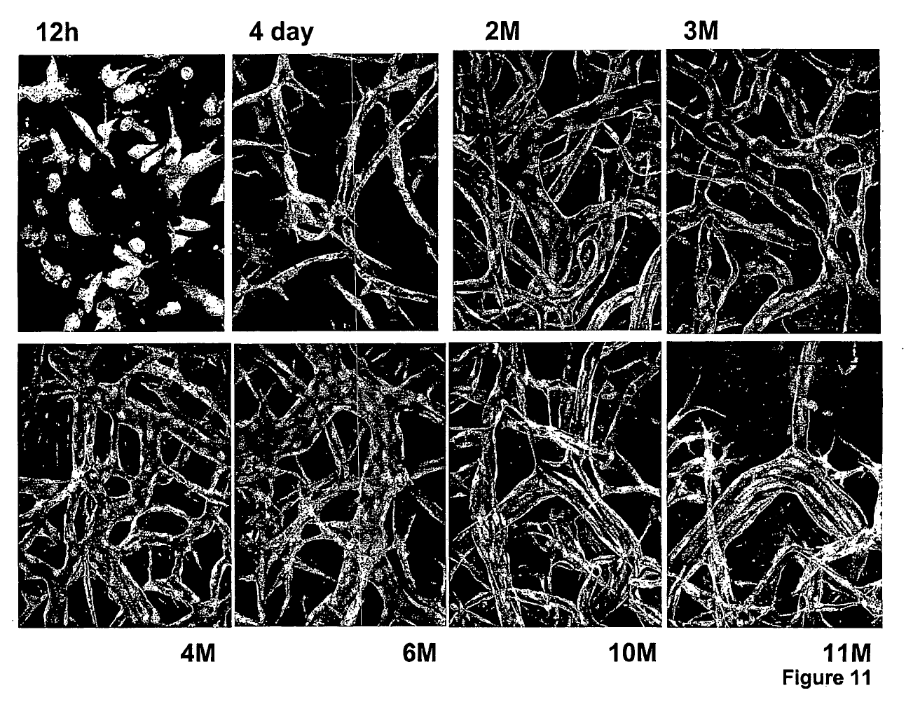

We have also created long-lasting vascular networks in vivo by co-implanting vascular endothelial cells and mesenchymal precursor cells. These vessels are stable and functional for up to one year (FIG. 11).

To form stable vessels, human umbilical vein endothelial cells (HUVECs) and 10T1/2 mesenchymal precursor cells were seeded in a three-dimensional (3-D), fibronectin-type I collagen gel. HUVECs were provided by Dr. F. W. Luscinskas (Brigham & Women's Hospital, Harvard Medical School, Boston, Mass.) and maintained in EGM medium (Cambrex Bio Science, Baltimore, Md.). C3H10T1/2 (10T1/2) (American Type Culture Collection, Manassas, Va.) were grown and maintained in Dulbecco's modified Eagle's medium (DMEM; Mediatech, Hemdon, Va.) supplemented with 10% fetal bovine serum (FBS), penicillin (100 units/ml) and streptomycin (100 mg/ml) (both from Life Technologies, Inc.). 1×10⁶ HUVECs (HUVEC-alone group) or 8×10⁵ HUVECs and 2×10⁵ of 10T1/2 (HUVEC+10T1/2 co-implantation group) were suspended in 1 ml solution of rat-tail type 1 collagen (1.5 mg/ml) and human plasma fibronectin (90 mg/ml) (both from Collaborative Biomedical Products, Bedford, Mass.) in 25 mM Hepes (Sigma) buffered EGM medium at 4° C. pH was adjusted to 7.4 using 0.1M NaOH (Fisher Science, N.J.). The cell suspension was pipetted into 12-well plates (Falcon) and warmed to 37° C. for 30 minutes to allow polymerization of collagen. Each solidified gel construct was covered by 1 ml of warmed EGM medium. After one day of culture in 5% CO₂, circular disk-shape pieces of the construct (4-mm diameter) were isolated by a skin puncher and implanted into the cranial windows. The HUVEC vs. 10T1/2 cell ratio of 4:1 was based on our preliminary studies. When we used a higher concentration of 10T1/2 cells, the implanted gels often shrank due to overgrowth of 10T1/2 cells, and the onset of perfusion was delayed. Conversely, when we used a lower concentration of 10T1/2 cells, viability and capillary formation were poor in long-term culture (˜2weeks; in vitro).

The 10T1/2 cells differentiated into mural cells by heterotypic interaction with endothelial cells (Orlidge et al, J. Cell Biol. 105:1455-1462, 1987; Hirschi et al., J. Cell Biol., 141:805-814; Darland et al., Angiogenesis 4:11-20, 2001). To permit continuous in vivo monitoring of the engineered vascular networks, we implanted these 3-D constructs in mice bearing transparent windows (Jain et al., Nat. Rev. Cancer 2:266-276, 2002). To track implanted HUVECs, we introduced the gene for enhanced green fluorescent protein (EGFP; Schechner et al., supra). Immediately after implantation, the HUVECs exhibited round or spindle-shape morphologies (FIG. 11, 12 hours). Intracellular vacuoles were also observed in some HUVECs (arrow heads). Thereafter, HUVECs formed long interconnected tubes with many branches (FIG. 11, Day 4). Large vacuoles in the tubes resemble the lumens of capillaries (arrows). Similar structural changes were reported in an in vitro 3-D angiogenesis assay (Yang et al., Am. J. Pathol. 155: 887-896, 1999).

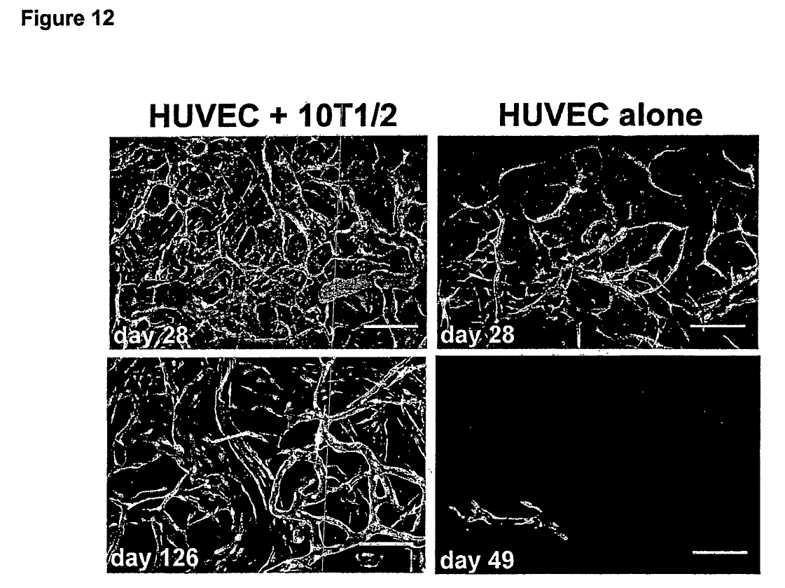

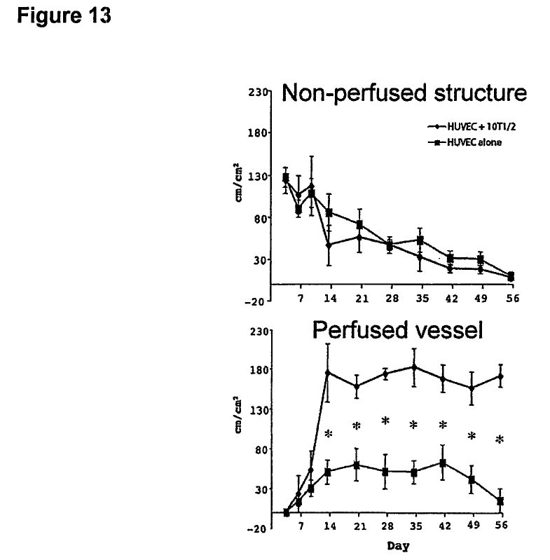

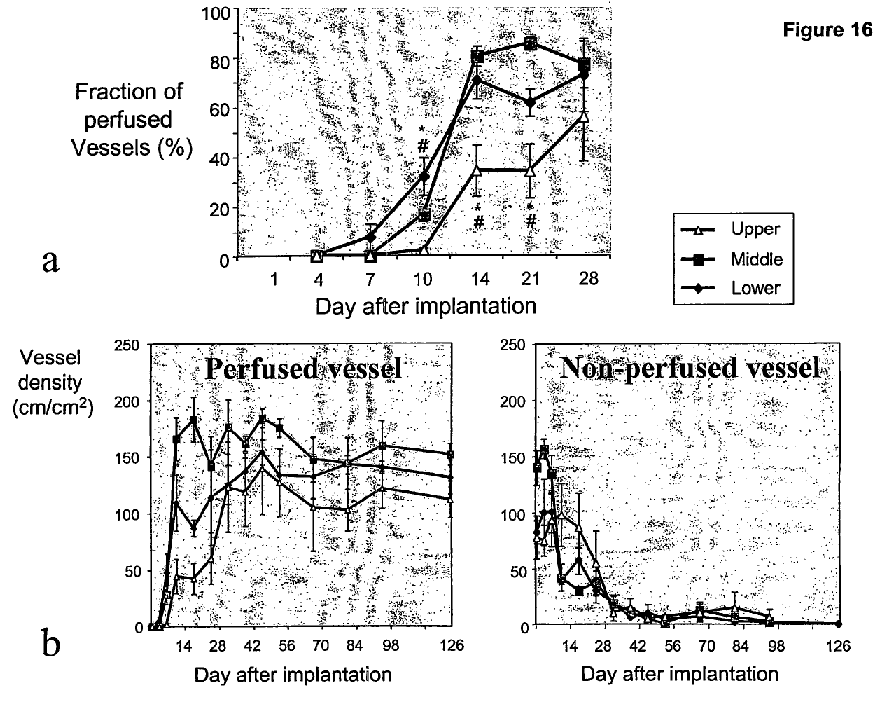

There were no apparent differences between the HUVEC-alone group and the co-implantation (HUVEC+10T1/2) group during these early morphological changes. However, subsequent to the initial formation of a fine, mesh-like structure with no perfusion (FIG. 12), the HUVECs connected to the circulatory system of the mouse and became perfused (FIGS. 12 and 13). The number of perfused vessels increased rapidly in the first two weeks and thereafter became stable, while non-perfused vessels gradually decreased in number and eventually disappeared (FIG. 13, FIGS. 16A and 16B). Perfused blood vessels associated with 10T1/2 cell-derived mural cells became stable for the long term, two to four weeks after implantation. In contrast, the 3-D constructs made of HUVECs alone showed minimal perfusion and disappeared within 60 days, despite similar initial appearance (FIGS. 12 and 13). Mural cells derived from 10 T1/2 cells stabilized blood vessels (Orlidge et al., supra), and thus they did not increase or decrease in number.

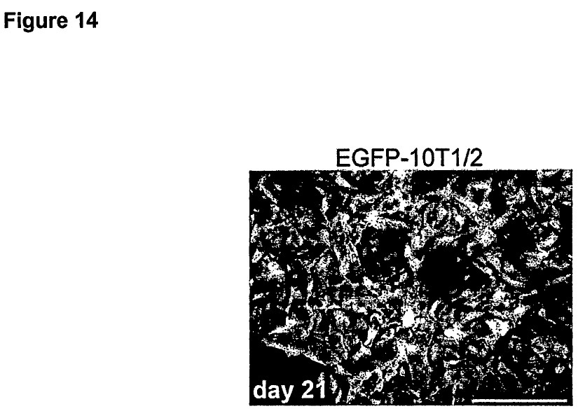

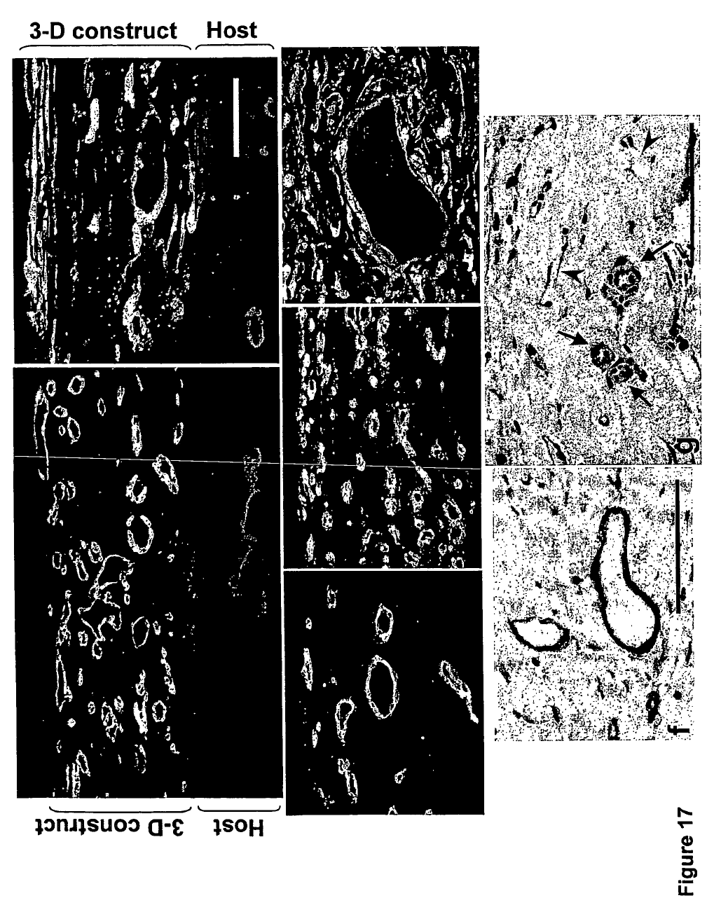

10T1/2 cells were labelled genetically to confirm their incorporation into the vessel wall by in vivo microscopy (FIG. 14) and immunohistochemistry (FIGS. 17A-17E). Functional vessels were lined by cells which were positive for human CD31 (an endothelial cell marker) and were covered by 10T1/2 cells that expressed α-smooth muscle actin (a mural cell marker). Generally, implanted 10T1/2 cells became mural cells of the engineered vessels. However, at the interface of implanted gel and host tissue, 10T1/2 cells occasionally infiltrated outside the gel and covered host vessels. At the same time, some engineered vessels without coverage of GFP-10T1/2 cells were observed under multi-photon laser-scanning microscopy.

There were also many 10T1/2 derived cells which did not associate with blood vessels in the gel (FIGS. 17B and 17E). The major difference between vessel-associated and non-vessel associated 10T1/2 cells was the expression of the mural cell marker a-smooth muscle actin. In FIGS. 17B and 17E, the vessel-associated 10T1/2 cells express α-SMA and became orange. This is contrast with abnormal blood vessels (e.g., in tumors) where many α-SMA-positive cells with loose or no association with endothelial cells are present (Morikawa et al., Am. J. Pathol., 160:985-1000, 2002). Host mural cells did line the outsides of vessels to some extent in the HUVEC alone gels (FIG. 17F). However, the survival of engineered vessels in HUVEC-alone gels is a rare event. Delayed recruitment of mural cells from the underlying host tissue resulted in the regression of the majority of HUVEC-alone-engineered vessels. On the other hand, in the co-culture gels, the abundance of mural precursors allowed efficient recruitment and mural cell investment of the engineered vessels. Hence, these vessels became stable and functional.

As in normal microcirculation, arteriolar and venular sides of the engineered vessels were readily identified by their morphology and blood flow pattern, and their identity was confirmed by histology (FIG. 17G).

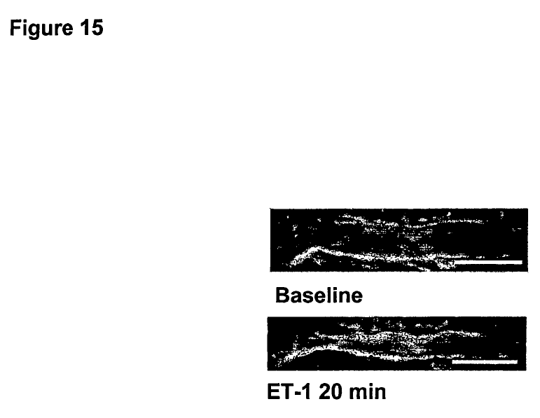

We tested the functionality of the mural cells in these engineered vessels by local administration of a vasoconstrictor, endothelin-1, which prompted stronger constriction of the engineered arterial vessels than those in HUVEC-alone gel (FIG. 15 and Table 1).

Arteriolar contractility was determined by vasoactive response to endothelin-1 (ET-1). After careful removal of the cover glass, the cranial window was superfused with warm PBS. For vessel contrast enhancement, 100 μl of 1% rhodmine-dextran (MW 2 million) was injected intravenously. The engineered vessels were monitored by single photon fluorescence intravital microscopy using a 20× water-immersion objective. After the baseline measurements, superfusate was replaced with 100 nM ET-1 in PBS. Then, the same regions were repeatedly monitored over 30 minutes. 100 nM ET-1 was used for the study of arterial contractility based on the dosage reported in the literature. For example, 100-1,000 nM ET-1 was locally administrated to observe pulmonary arteriolar vasoconstriction (Roberts et al., Microcirculation 5:289, 1998) and 100 nM ET-1 was used to determine contractility of the aortic ring (Flamant et al., FASEB J 17:327-329, 2003).

Arterioles and venules were distinguished by their morphology and flow pattern in vivo. Arterioles branch out from larger vessels and have faster flow rate and smaller diameter. Venules, on the other hand, merge into larger vessels and have slower flow rate and larger diameter. Histological characteristics of arterioles and venules were also confirmed using H&E staining (FIG. 17G). Arterioles have a thicker vessel wall with circumferential mural cells whereas venules have a thin layer of mural cells.

TABLE 1
Arteriolar contractility of tissue engineered vessels.

| | Time after ET-1 superfusion | | | |
|---|---|---|---|---|
| | 5 min | 10 min | 15 min | 20 min |
| HUVEC + 10T1/2 | 36.3 ± 6.9*% | 54.3 ± 5.6*% | 66.2 ± 6.5*% | 71.0 ± 7.1*% |
| HUVEC alone | 16.0 ± 4.7% | 29.1 ± 6.8% | 36.5 ± 7.5% | 43.8 ± 9.6% |

Data are expressed as mean ± SEM.
*p < 0.05 as compared with corresponding HUVEC alone group.

It is noteworthy that the response to ET-1 was variable in both groups, presumably due to the different degree of mural cell coverage and baseline vessel tone. For vessel constriction studies, we focused on arterioles or arteriole-like vessels, since these are the vessels that predominantly respond to vasoactive agents in a normal tissue. In fact, the diameter of capillary and venules did not change appreciably after ET-1 superfusion. In the co-culture gels (n=6, Day 44), the range of arteriolar constriction was 46-87% with an average of 71.0% at 20 minutes. On the other hand, vessel constriction in HUVEC-alone gels (n=6, Day 44) ranged between 19 and 86% with an average of 43.8% at 20 min. We also determined the contractility of engineered arterioles at Day 294 after implantation. Arterioles in the aged gel constricted by 40-60% (average maximum constriction 54%) in response to 100 nM ET-1.

Vascular permeability to albumin was also determined using tetramethyrhodamine-conjugated-BSA and intravital microscopy, using our previously published method (Yuan et al., Microvasc. Res. 45:269-289, 1993). Briefly, mice were injected with a bolus (100 μl) of 1% tetramethylrhodamine-labeled bovine serum albumin (Molecular Probes, Eugene, Oreg.) in saline via the tail vein. Fluorescence intensity of the tissue was measured every two minutes for a total of 20 minutes by a photomultiplier (9203B, EMI, Rockaway, N.J.) using a 20× objective lens. The effective vascular permeability (P) was calculated as follows: P=(1-HT) V/S {1/(I₀-I_{b})*dI/dt+1/K} where I is the average fluorescence intensity of the whole image, I₀ is the value of I immediately after the filling of all vessels by Rho-BSA and I_{b} is the background fluorescence intensity. HT is the average hematocrit. V and S are the total volume and surface area of vessels within the tissue volume covered by the surface image, respectively. The time constant of BSA plasma clearance (K) was 9.1×10³ S.

The vascular permeability of the engineered vessels was higher than that of normal quiescent vessels, but was in the lower range of vessels induced by various angiogenic molecules (Table 2).

TABLE 2
Vascular permeability of tissue engineered vessels
and other vessels in the mouse cranial window.

| | | | | Type I |
|---|---|---|---|---|
| | | Control | | collagen |
| HUVEC + | HUVEC- | cranial | | gel with |
| 10T1/2 | alone | window¹ | Tumours†^{1,2} | VEGF^{3,4} |
| 1.57 ± 0.06 × | 1.51 ± 0.17 × | 0.3˜0.6^{#} × | 2.9˜3.9^{#} × | 2.5˜4.9^{#} × |
| 10⁻⁷ cm/s | 10⁻⁷ cm/s | 10⁻⁷ cm/s | 10⁻⁷ cm/s | 10⁻⁷ cm/s |

Vascular permeability of engineered vessels was determined at Day 36 when the perfused vessels in both co-implantation construct and HUVEC-alone construct are relatively stable (FIG. 12). Data are expressed as mean ± SEM (n = 2 each).
^{#}Range of average values from literature.
†Includes LS174T human colon adenocarcionoma, U87 human glioma, MCaIV murine mammary carcinoma.
¹Monsky et al., Cancer Res 59: 4129-4135, 1999
²Yuan et al., Cancer Res 54: 4564-4568, 1994
³Dellian et al., Am J Pathol 149: 59-72, 1996
⁴Fukumura et al., PNAS 98: 2604-2609, 2001

Methods for Constructing Engineered Microvascular Scaffolds

Using preadipocytes and mesenchymal cell precursors to stabilize and enhance artificially generated blood vessel networks provides improved artificially engineered tissues having a functional microvascular scaffold. Blood or lymph vascular endothelial cells or endothelial precursor cells are used to prepare engineered blood and lymph vessels. Blood or lymph vascular endothelial cells form the primary layer of the engineered vessel; such cells are obtained, for example, from established endothelial cell lines, freshly prepared primary culture endothelial cells, or endothelial cells generated from stem cells in vitro. Sources of such embryonic stem cells include, but are not limited to, bone marrow derived stem cells, cord blood derived cells, HUVEC, lymphatic endothelial cells, embryonic stem cells, and endothelial pregenitor cells.

Cells that are useful for stabilizing the engineered vessels include perivascular cells (e.g., pericytes); vascular smooth muscle cells; mesenchymal precursor cells, (e.g., embryonic stem cells, 10T1/2 cells, preadipocytes, (e.g., TA1, 3T3-L1, 3T3-F442A, or Ob17) fibroblasts; murine embryonic fibroblasts; fibroblast cell lines (e.g., NIH 3T3, Swiss 3T3, BalbC 3T3); and tumor activated stromal cells (e.g., GFP positive cells isolated from tumors grown in VEGF-GFP mice, EF1a-GFP mice, or Tie2-GFP mice).

The interstices within the engineered microvascular networks can be filled with parenchymal cells from virtually any organ. Because many cell-types can be expanded in vitro, grafts can be made using a limited number of cells (e.g., 100, 500, 1000, 10,000, 100,000, 1,000,000, 10,000,000, or 100,000,000), which represent a small percentage (e.g., 0.0001%, 0.001%, 0.005%, 0.01%, 0.05%, 0.10%, 1.0%, 2.0%, or 5.0%) of the cells present in a naturally-occurring tissue or organ. Exemplary parenchymal cells for organogenesis include, hepatocytes, myocytes (e.g., cardiac or skeletal muscle myocytes), keratinocytes, osteocytes, chondrocytes, islet cells, nerve cells, astrocytes, glial cells from the central or peripheral nervous system, preadipocytes derived from fat or breast tissue, and adipocytes. Such cells might be obtained from the intended implant recipient (an autograft), from a donor (allogeneic graft), or from a cell line. One particular advantage of autografts is that the grafted tissue does not induce an immune response because the grafted cells are recognized as self (Heath et al., Trends Biotechnol, 18:17-19, 2000). In other embodiments, such cells might be obtained from a mammal of a different species (e.g., pig or primate).

Cell Isolation

One aspect of the invention pertains to a replacement organ that includes an engineered microvascular scaffold. The replacement organs may be derived from the recipient's own tissue, derived from a different individual of the same species, or derived from a mammalian species that is different from the recipient (e.g., pig or primate).

Cells can be isolated from a number of sources, for example, from biopsies or autopsies using standard methods. The isolated cells are preferably autologous cells obtained by biopsy from the subject. The cells from biopsy can be expanded in culture. Cells from relatives or other donors of the same species can also be used with appropriate immunosuppression. Methods for the isolation and culture of cells are discussed in Fauza et al. (J. Ped. Surg. 33, 7-12, 1998)

Cells are isolated using techniques known to those skilled in the art. For example, the tissue or organ can be disaggregated mechanically and/or treated with digestive enzymes and/or chelating agents that weaken the connections between neighboring cells making it possible to disperse the tissue into a suspension of individual cells without appreciable cell breakage. Enzymatic dissociation can be accomplished by mincing the tissue and treating the minced tissue with digestive enzymes (e.g., tlypsin, chymotrypsin, collagenase, elastase, hyaluronidase, DNase, pronase, and dispase). Mechanical disruption can be accomplished by scraping the surface of the organ, the use of grinders, blenders, sieves, homogenizers, pressure cells, or sonicators. For a review of tissue disruption techniques, see Freshney, (Culture of Animal Cells. A Manual of Basic Technique, 2d Ed., A. R. Liss, Inc., New York, Ch. 9, pp. 107-126, 1987)

Preferred cell types include, without limitation, adipocytes, preadipocytes, urothelial cells, mesenchymal cells, especially smooth or skeletal muscle cells, myocytes (muscle stem cells), mesenchymal precursor cells, cardiac myocytes, fibroblasts, chondrocytes, fibromyoblasts, ectodermal cells ductile cells, and skin cells, hepotocytes, islet cells, cells present in the intestine, parenchymal cells, other cells forming bone or cartilage (e.g., osteoblasts), and nerve cells.

Once the tissue has been reduced to a suspension of individual cells, the suspension can be fractionated into subpopulations. This may be accomplished using standard techniques (e.g., cloning and positive selection of specific cell types or negative selection, i.e., the destruction of unwanted cells). Selection techniques include separation based upon differential cell agglutination in a mixed cell population, freeze-thaw procedures, differential adherence properties of the cells in the mixed population, filtration, conventional and zonal centrifugation, unit gravity separation, countercurrent distribution, electrophoresis and fluorescence-activated cell sorting. For a review of clonal selection and cell separation techniques, see Freshney, Culture of Animal Cells. A Manual of Basic Techniques, 2d Ed., A. R. Liss, Inc., New York, Ch. 11 and 12, pp. 137-168, 1987).

Cell fractionation may be useful when the donor has a disease, such as cancer. Isolated cells can be cultured in vitro to increase the number of cells available for transplantation. The use of allogenic cells, and more preferably autologous cells, is preferred to prevent tissue rejection. However, if an immunological response does occur in the subject after implantation of the engineered organ, the subject may be treated with immunosuppressive agents, such as cyclosporin or FK506, to reduce the likelihood of rejection.

Isolated cells may be transfected. Useful genetic material may be, for example, genetic sequences that are capable of reducing or eliminating an immune response in the host. For example, the expression of cell surface antigens such as class I and class II histocompatibility antigens may be suppressed. This may allow the transplanted cells to have reduced chance of rejection by the host. In addition, transfection could also be used for gene delivery. The cell-substrate construct can carry genetic information required for the long-term survival of the host or the artificial organ or for detecting or monitoring the cells. In one example, the cell or cells of the microvascular scaffold are genetically modified to express a bioactive molecule that promotes angiogenesis. In another example, the cell or cells of the microvascular scaffold are genetically modified to expresses a fluorescent protein marker. Exemplary markers include GFP, EGFP, BFP, CFP, YFP, and RFP. The cell-substrate construct can also carry genetic information required for promoting or maintaining angiogenesis. Transfection may be used for transient gene expression or stable gene expression by incorporation of the gene into the host cell.

Isolated cells can be normal or genetically-engineered to provide additional or normal function. Methods for genetically engineering cells with viral vectors such as retroviral vectors or other methods known to those skilled in the art can be used. These include using expression vectors which transport and express nucleic acid molecules in the cells (see, for example, Goeddel et al., (Gene Expression Technology: Methods in Enzymology 185, Academic Press, San Diego, Calif., 1990).

Vector DNA is introduced into prokaryotic or eukaryotic cells via conventional transformation or transfection techniques. Suitable methods for transforming or transfecting host cells can be found in Sambrook et al. (Molecular Cloning: A Laboratory Manual, 2nd Edition, Cold Spring Harbor Laboratory press (1989)), and other laboratory textbooks.

Methods for Making Cell Suspensions

Isolated parenchymal cells are mixed with a matrix and injected directly, or cultured for a time with a suitable matrix polymer that may or may not have an existing microvascular scaffold. The polymer is dissolved in an aqueous solution, preferably a 0.1 M potassium phosphate solution, at physiological pH, to a concentration forming a polymeric hydrogel. The isolated cells, including an adipocyte, preadipocyte, mesenchymal precursor cell or a mesenchymal cell; with an endothelial cell and a parenchymal cell, are suspended in the polymer solution to a concentration of between 0.5 and 500 million cells/ml, preferably between 1 and 50 million cells/ml, and most preferably between 5 and 10 million cells/ml.

Cells are cultured on or embedded in a matrix, or injected into a matrix already implanted at the desired site. Desirably, the matrix is a pliable, non-toxic, injectable porous template that allows for vascular growth. The pores should allow for vascular growth and the injection of cells without damage to the cells or to the patient. These are generally interconnected pores in the range of between approximately 100 and 300 microns. The matrix should be shaped to maximize surface area, to allow adequate diffusion of nutrients and growth factors to the cells and to allow the growth of new blood vessels and connective tissue. A porous structure that is resistant to compression is preferred. The matrix configuration is dependent on the tissue which is to be treated, repaired, or produced. In one example the matrix includes type I collagen and plasma fibronectin.

Methods for Repairing Damaged Tissues and Organs

The invention features methods of repairing diseased or damaged tissues and organs. Cells (e.g., preadipocytes, adipocytes having or not having a genetic modification, perivascular cells, vascular smooth muscle cells, mesenchymal precursor cells, mesenchymal cells, and fibroblasts) are administered to a damaged or diseased tissue or organ. These cells induce blood vessel formation and a functional blood vessel network in the damaged tissue or organ thereby increasing blood supply to the organ, improving organ biological function, and increasing parenchymal cell proliferation. Optionally, endothelial cells or endothelial cell precursors may also be administered. These methods may stabilize a damaged tissue or organ in a patient on a transplantation waiting list; or the methods may repair a damaged or diseased tissue or organ, thereby obviating the need for transplantation. Methods for repairing damaged tissue or organs may be carried out either in vitro, in vivo, or ex vivo.

Methods for Organ or Tissue Transplantation

The invention features improved methods for organ or tissue transplantation. Cells (e.g., preadipocytes, adipocytes having or not having a genetic modification, perivascular cells, vascular smooth muscle cells, mesenchymal precursor cells, mesenchymal cells, and fibroblasts) are administered to a tissue or organ (donor organ or engineered organ) prior to, during, or after transplantation. Optionally, endothelial cells or endothelial precursor cells may also be administered. The administration of one or more of these cell-types enhances blood vessel formation and a functional blood vessel network in the tissue or organ, increases infiltration of the graft by host blood vessels, and improves the integration of the graft into the host's circulatory system.

Methods for Producing Engineered Tissues or Organs

The invention features methods of producing engineered replacement organs or tissues comprising a functional microvasculature. Cells (e.g., preadipocytes, adipocytes, mesenchymal cell precursors, mesenchymal cells, blood and/or lymph vascular endothelial cells, pericytes, vascular smooth muscle cells, and tumor activated stromal cells) are preferably cultured in the presence of a matrix (e.g., a synthetic polymer based matrix, decellularized skin or other tissue source; collagen or other extracellular matrix gel) as described herein. Optionally, the interstices of the microvascular scaffold are filled with parenchymal cells, derived from virtually any organ of interest, to generate an engineered tissue or organ. Parenchymal cells may be introduced prior to, during, or after formation of the microvascular scaffold. Methods for producing an engineered tissue or organ may be carried out either in vitro, in vivo, or ex vivo.

Engineered Tissues and Organs

Organs that can be produced using the methods of the invention include, but are not limited to, the bladder, brain, nervous tissue, glial tissue, esophagus, fallopian tube, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, and cartilage. Such organs are generated, for example, by culturing a pre-adipocyte, adipocyte, mesenchyrnal precursor, or mesenchymal cell and an endothelial cell to produce a functional microvascular scaffold.

Parenchymal cells are introduced to the culture, and allowed to fill the interstitial spaces within the microvasculature. Alternatively, parenchymal cells are introduced to the matrix before, during, or after formation of the microvascular scaffold. Most preferably, an engineered organ comprises (i) an adipocyte, preadipocyte, or a mesenchymal precursor cell, (ii) an endothelial cell, and a (iii) parenchymal cell embedded in a matrix or cultured on the surface of a matrix. Optionally, an engineered organ may comprise only parenchymal cells and an adipocyte, preadipocyte, or a mesenchymal precursor cell.

Engineered organs are useful for the treatment of a variety of diseases or disorders. In particular, an engineered organ comprising insulin-producing cells, adipocytes, preadipocytes, mesenchymal cells, or mesenchymal precursor cells, and endothelial cells, is administered to a patient for the treatment or prevention of diabetes; oligodendroglial precursor cells, adipocytes, preadipocytes, mesenchymal cells, or mesenchymal precursor cells, and endothelial cells are administered for the treatment or prevention of multiple sclerosis. For the treatment or prevention of endocrine conditions, engineered organs that produce a hormone, such as a growth factor, thyroid hormone, thyroid-stimulating hormone, parathyroid hormone, steroid, serotonin, epinephrine, or norepinephrine may be administered to a mammal. Additionally, an engineered organ comprising epithelial cells, adipocytes, preadipocytes, or mesenchymal precursor cells, and endothelial cells are administered to repair damage to the skin, or to the lining of a body cavity or organ, such as a lung, gut, or urogenital tract. A replacement liver is generated by culturing a hepatocyte and an adipocyte, preadipocyte, or mesenchymal precursor cell, and an endothelial cell. It is also contemplated that parenchymal cells having a microvascular scaffold are administered to a mammal to treat damage or deficiency of cells in an organ, muscle, or other body structure, or to form an organ, muscle, or other body structure. Desirable organs include the bladder, brain, nervous tissue, glial tissue, esophagus, fallopian tube, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, and cartilage. Optionally, a replacement organ comprises only parenchymal cells and an adipocyte, preadipocyte, or a mesenchymal precursor cell.

Parenchymal cells are also combined with a matrix and a microvascular scaffold to form a tissue or organ in vitro or in vivo that may be used to repair or replace a tissue or organ in a recipient mammal. For example, parenchymal cells are cultured in vitro or in vivo in the presence of a matrix and a microvascular scaffold to produce a tissue or organ that is transplanted into a mammal.

Engineered Bone or Cartilage

Exemplary transplantation methods of the present invention also include repairing or replacing bone or cartilaginous tissue. Traditional bone or cartilage tissue engineering methods can be improved by administering preadipocytes, adipocytes not having a genetic modification, perivascular cells, vascular smooth muscle cells, mesenchymal precursor cells, mesenchymal cells, and fibroblasts to the damaged or diseased bone or cartilage in vivo or to a bone or cartilage transplant tissue before, during, or after the transplant tissue is administered to a mammal. Preferably, endothelial cells or endothelial precursor cells are also administered to enhance angiogenesis. Traditional bone and cartilaginous tissue reconstruction methods are described, for example, in U.S. Pat. Nos. 6,197,061; 6,197,586; 6,228,117; 6,419,702; and 6,451,060. Engineered bone is useful for the treatment of a variety of diseases or disorders, including arthritis, cancer, congenital defects of bone or cartilage such as worn or torn cartilage in joint linings (e.g., knee joint, hip joint, and temporomandibular joint) and trauma.

It is known that connective-tissue cells, including fibroblasts, cartilage cells, and bone cells, can undergo radical changes of character. Thus, as explained by Alberts et al., (Molecular Biology of the Cell 2nd Ed., pp. 987-988, 1989), a preparation of bone matrix may be implanted in the dermal layer of the skin and some of the cells there are converted into cartilage cells and others into bone cells.

A great variety of materials are useful as matrices for this purpose. For example, materials such as collagen gels, poly(D,L-lactide-co-glycolide (PLGA) fiber matrices, polyglactin fibers, calcium alginate gels, polyglycolic acid (PGA) meshes, and other polyesters such as poly-(L-lactic acid) (PLLA) and polyanhydrides are among those suggested. Matrices can include materials that are non-biodegradable or biodegradable. Desirably, biodegradable materials will degrade over a time period of less than a year, more preferably less than six months.

Methods for treating connective tissue disorders using engineered cartilaginous or connective tissues are described, for example, in U.S. Pat. Nos.: 5,226,914; 5,041,138; 5,368,858; 5,632,745; 6,451,060; 6,197,586; and 6,197,061. In some embodiments, chondrocytes are cultured with (i) a preadipocyte, an adipocyte, a mesenchymal precursor cell, or a mesenchymal cell, and (ii) an endothelial cell or an endothelial precursor cell and then implanted. Surgical procedures related to bone tissue deficiencies vary from joint replacement or bone grafting to maxillo-facial reconstructive surgery. Such methods are known to the skilled artisan.

Engineered Soft Tissue

Traditional methods of soft tissue reconstruction, as described in U.S. Pat. No. 5,716,404, can be improved by administering preadipocytes, adipocytes not having a genetic modification, perivascular cells, vascular smooth muscle cells, mesenchymal precursor cells, mesenchymal cells, and fibroblasts to the soft tissue to be transplanted. For example, engineered soft tissue is useful for cosmetic surgery or for reconstruction of the breast, face, or other body part after cancer surgery or trauma. Preferably, engineered soft tissues comprises (i) a soft tissue parenchymal cell (e.g., a skin cell, subcutaneous fat cell, muscle sheath (fascia) cell, muscle cell, or adipocyte), (ii) a preadipocyte, adipocyte not having a genetic modification, perivascular cell, vascular smooth muscle cells, mesenchymal precursor cell, and fibroblast, and (iii) an endothelial cell or endothelial precursor cell. Optionally, an engineered soft tissue comprises only soft tissue parenchymal cells and preadipocytes, adipocytes having or not having a genetic modification, perivascular cells, vascular smooth muscle cells, mesenchymal precursor cells, and fibroblasts. For example, a breast is generated by culturing a breast parenchymal cell, a pre-adipocyte, adipocyte, mesenchymal cell, or mesenchymal precursor cell, and an endothelial cell to produce a functional microvascular scaffold. Parenchymal cells may be introduced to the culture, prior to, during, or after formation of the microvascular scaffold. The parenchymal cells are allowed to fill the interstitial spaces within the microvasculature.

For soft tissue reconstruction, the matrix, which is mixed with cells (e.g., (i) preadipocytes, adipocytes not having a genetic modification, perivascular cells, vascular smooth muscle cells, mesenchymal precursor cells, and fibroblasts, (ii) endothelial cells, and (iii) parenchymal cells), may form a hydrogel. A hydrogel is defined as a substance formed when an organic polymer (natural or synthetic) is cross-linked via covalent, ionic, or hydrogen bonds to create a three-dimensional open-lattice structure that entraps water molecules to form a gel. Examples of materials that can be used to form a hydrogel include polysaccharides (e.g., alginate), polyphosphazenes, and polyacrylates (e.g., hydroxyethyl methacrylate). Other materials that can be used include proteins (e.g., fibrin, collagen, fibronectin) and polymers (e.g., polyvinylpyrrolidone), and hyaluronic acid.

In general, these polymers are at least partially soluble in aqueous solutions, (e.g., water) buffered salt solutions, or aqueous alcohol solutions, which have charged side groups, or monovalent ionic salts thereof. Examples of polymers with acidic side groups that can be reacted with cations are poly(phosphazenes), poly(acrylic acids), poly(methacrylic acids), copolymers of acrylic acid and methacrylic acid, poly(vinyl acetate), and sulfonated polymers (e.g., sulfonated polystyrene). Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

Examples of polymers with basic side groups that can be reacted with anions are poly(vinyl amines), poly(vinyl pyridine), poly(vinyl imidazole), and some imino substituted polyphosphazenes. The ammonium or quaternary salt of the polymers can also be formed from the backbone nitrogens or pendant imino groups. Examples of basic side groups are amino and imino groups.

Alginate can be ionically cross-linked with divalent cations in water at room temperature to form a hydrogel matrix. Additional methods for the synthesis of the other polymers described above are known to those skilled in the art (see, for example, Concise Encyclopedia of Polymer Science and Polymeric Amines and Ammonium Salts, E. Goethals, editor, Pergamen Press, Elmsford, N.Y. 1980). Many polymers, such as poly(acrylic acid), are commercially available.

Synthetic Polymers

Synthetic polymers can also be used to form a matrix, and are preferred for reproducibility and controlled release kinetics. Synthetic polymers that can be used include bioerodible polymers such as poly(lactide), poly(glycolic acid), poly(lactide-co-glycolide), poly(caprolactone), polycarbonates, polyamides, polyanhydrides, polyamino acids, polyortho esters, polyacetals, polycyanoacrylates and degradable polyurethanes, and non-erodible polymers such as polyacrylates, ethylene-vinyl acetate polymers and other acyl substituted cellulose acetates and derivatives thereof, non-erodible polyurethanes, polystyrenes, polyvinyl chloride, polyvinyl fluoride, poly(vinyl imidazole), chlorosulphonated polyolifins, polyethylene oxide, polyvinyl alcohol, teflon.RTM., and nylon. Non-degradable materials can also be used to form the matrix.

One preferred non-degradable material for implantation of a matrix is a polyvinyl alcohol sponge, or alkylation or acylation derivatives thereof (e.g., ester derivatives). including esters. A non-absorbable polyvinyl alcohol sponge is available commercially as Ivalon.TM. from Unipoint Industries. These materials are all commercially available.

Preferred polymers for use in the matrix have mechanical and biochemical properties that enhance viability and proliferation of transplanted cells, tissues, or organs.

Synthetic Degradable Polymer Matrices

Synthetic degradable polymer matrices have been proposed as a means of tissue reconstruction and repair. The matrix serves as both a physical support and an adhesive substrate for isolated cells during in vitro culturing and subsequent in vivo implantation. Matrices are used to deliver cells to desired sites in the body, to define a potential space for engineered tissue, and to guide the process of tissue development. Cell transplantations on matrices are useful for the regeneration of tissues and organs (e.g., skin, nerve, liver, pancreas, cartilage and bone tissue) using various biological and synthetic materials.

Engineered Tissue and Organ Transplantation

A donor organ, donor cell, engineered tissue, or engineered organ is transplanted into a patient (e.g., a human or mammal) for the treatment or stabilization of a condition, disease, or disorder using standard methods known to the skilled artisan. For example, methods for transplanting engineered blood vessels are described by Hibino et al., (Kyobu Geka 55:368-73, 2002); methods for engineered ventricular tissue transplantation are described by Krupnick et al., (J. Heart Lung Transplant 21(2):233-43, 2002) and Nishina et al. (Clin. Exp. Pharmacol. Physiol. 29:728-30, 2002); methods for implanting engineered skin are described by Donati et al., (Biol. Neonate 80:273-6, 2001); methods for implanting a tissue-engineered stomach are described by Hori et al., (ASAIO J. 47:206-210, 2001); methods for implanting an engineered bladder are described by Schoeller et al. (J. Urol. 165:980-985, 2001) and Oberpenning (Nat Biotechnol 17:149-55, 1999); methods for three-dimensional skeletal muscle tissue-engineering are described by Saxena et al., (Biomed. Mater. Eng. 11(4):275-281, 2001); methods for adipose tissue implantation are described by Patrick (Semin. Surg. Oncol. 19:302-311, 2000; methods for implanting engineered liver support devices are described by Filipponi et al., (Clin Exp Pharmacol Physiol 29:728-730, 2002) and Makowka (Surgery 88:244-253, 1980); methods for liver transplantation are described by Kalayoglu (J. Am. Coll. Surg. 182(5):381-387, 1996); methods for implanting an engineered kidney are described by Ota et al., (Laboratory & Clinical Medicine. 140(1):43-51, 2002); and methods for lung and heart transplantation are described in LeGal et al., (Ann. Thorac. Surg. 49:840-844, 1990).

Additional methods of removing, storing, and transplanting an organ are standard in the art, and are described, for example, in U.S. Pat. No. 5,693,462.

Methods for Evaluating Therapeutic Efficacy

Methods of the invention are useful for treating or stabilizing in a patient (e.g., a human or mammal) a condition, disease, or disorder affecting a tissue or organ. Therapeutic efficacy is optionally assayed by measuring, for example, the biological function of the treated or transplanted organ (e.g., bladder, bone, brain, breast, cartilage, esophagus, fallopian tube, heart, pancreas, intestines, gallbladder, kidney, liver, lung, nervous tissue, ovaries, prostate, skeletal muscle, skin, spinal cord, spleen, stomach, testes, thymus, thyroid, trachea, ureter, urethra, urogenital tract, and uterus). Such methods are standard in the art and exemplary methods follow. Bladder function is assayed by measuring urine retention and excretion. Brain, spinal cord, or nervous tissue function is assayed by measuring neural activity (e.g., electrical activity). Esophageal function is assayed by measuring the ability of the esophagus to convey food to the stomach. Fallopian tube function is assayed by injecting radiopaque agents. Heart function is assayed by electrocardiogram. Pancreatic function is assayed by measuring insulin production. Intestinal function is assayed by measuring the ability of intestinal contents to pass through to the bowel, and may be evaluated using a barium enema or GI series. Gallbladder function is assayed using a gall bladder radionuclide scan. Kidney function is assayed by measuring creatinine levels, urine creatinine levels, or by clinical tests for creatinine clearance, or blood urea nitrogen. Liver function is assayed using liver function tests or a liver panel that measures liver enzyme levels, bilirubin levels, and albumin levels. Lung function is assayed using spirometry, lung volume, and diffusion capacity tests. Ovary function is assayed by measuring levels of ovarian hormones (e.g., follicle stimulating hormone). Prostate abnormality is assayed by measuring prostate specific antigen. Spleen function is assayed using a technetium scan or liver-spleen scan. Stomach function is assayed using a stomach acid test or by assaying gastric emptying. Testicular function is assayed by measuring levels of testicular hormones (e.g., testosterone). Other methods for assaying organ function are known to the skilled artisan and are described, for example, in the Textbook of Medical Physiology, Tenth edition, (Guyton et al., W.B. Saunders Co., 2000).

Preferably, a transplantation method of the present invention, increases the biological function of a tissue or organ by at least 5%, 10%, 20%, 40%, 50%, 60%, 70%, 80%, 90%, 100%, 150%, 200%, or even by as much as 300%, 400%, or 500%.

In addition, the therapeutic efficacy of the methods of the invention can optionally be assayed by measuring an increase in cell number in the treated or transplanted tissue or organ as compared to a corresponding control tissue or organ (e.g., a tissue or organ that did not receive treatment). Preferably cell number in a tissue or organ is increased by at least 5%, 10%, 20%, 40%, 60%, 80%, 100%, 150%, or 200% relative to a corresponding tissue or organ. Methods for assaying cell proliferation are known to the skilled artisan and are described in (Bonifacino et al., Current Protocols in Cell Biology Loose-leaf, John Wiley and Sons, Inc., San Francisco, Calif.).)

Alternatively, the therapeutic efficacy of the methods of the invention is assayed by measuring angiogenesis, blood vessel formation, blood flow, or the function of a blood vessel network in the tissue or organ receiving treatment as compared to a control tissue or organ (e.g., corresponding tissue or organ that did not receive treatment). A method that increases blood vessel formation (e.g., by at least 5%, 10%, 20%, 30%, 40%, 50%, 60%, 70%, 80%, 100%, 150%, or 200%, or even by as much as 300%, 400%, or 500% is considered to be useful in the invention. Methods for evaluating angiogenesis are standard in the art and are described herein.

Methods for Screening for Compounds with Angiogenic or Vasculogenic Activity

The engineered vessels and the angiogenesis-organogenesis and 3-D matrix models of angiogenesis described herein can be used to study the dynamic processes of angiogenesis, vessel maturation, and vessel remodeling. These models can be used to understand how endothelial cells assemble tube-like structures in vivo, how perivascular cells (pericytes, vascular smooth muscle cells) are recruited, and how the engineered vessels connect to pre-existing host vessels.

The engineered vessels and the models described herein can also be used to study the potential use of stem cell/progenitor cell for tissue blood vessel engineering. Endothelial cells can be derived from embryonic stem cells, bone marrow cells, and circulating progenitor cells. Perivascular cells can be derived from embryonic stem cells, mesenchymal precursors from embryo, bone marrow, and potentially circulating progenitor cells. The models described herein can be used to study the differentiation process of these precursor cells in vivo and can also be used to examine practical use of these cells for tissue engineering purpose.

Any of the engineered vessel models described herein can also be used to study the effects of any compounds that can promote or prevent angiogenesis or vasculogenesis or both. Non-limiting examples of compounds that can potentially mediate vessel maturation or remodeling in vivo or in vitro include VEGF family proteins and VEGFR family members, PDGFB and PDGFRβ, TGFβ and its down stream signaling pathway components, angiopoietins, components of the Tie2 pathway, ephrins, notch pathway signaling proteins, S1p1-EDG1, and NO and its downstream signaling pathway components (for a more detailed review of proteins involved in angiogenesis see Jain Nature Medicine 9:685-692, 2003). Compounds to be evaluated can be added to the animal or three-dimensional matrix models of angiogenesis and their effect on angiogenesis can be measured in the engineered blood vessel. Furthermore, titration studies of these compounds can be performed using the engineered vessels or the models described herein to determine optimal amounts of the compounds to induce or prevent angiogenesis or vasculogenesis in vivo or in vitro.

In one example, the effect of a PDGF-BB receptor antagonist was evaluated. The addition of Tyrphostin AG1295: 6,7-Dimethyl-2-phenylquinoxaline (Sigma; Kovalenko et al Cancer Res 54:6196-6114, 1994). AG1295 resulted in reduced stabilization of engineered vessels by 10T1/2 cells. Functional vessel density at days 14 and 35 in animals treated with AG1295 (12 mg/kg/day) was 65.9±30.7 cm/cm² (n=4) and 71.8±14.0 cm/cm² (n=4), respectively, whereas the vessel density in non-treated gel at the same days was approximately 180 cm/cm² (FIG. 11).

The temporal effect of various test compounds can also be determined. Proper alignment of blood vessels with endothelial cells and mural cells require local control (gradient) of key compounds. Furthermore, formation of functional units of blood vessels requires differentiation into different type of blood vessels such as arterioles, capillaries, and venules which have different morphological features and gene/molecule expression profiles. For example, addition of an exogenous compound may accelerate the initial step but may not be necessary for the development of mature vessels. Alternatively, addition of an exogenous compound may not affect the initial steps required for blood vessel formation but may be needed for differentiation into different types of blood vessels. The effects of test compounds in each of these processes can be determined using the methods to induce blood vessel formation or the angiogenesis models described herein. For example engineered blood vessels can be established in a mouse model for adipogenesis or in a three dimensional matrix model and the engineered blood vessels can be contacted with test compounds. The blood vessels treated with test compounds are then compared with untreated vessels and blood vessel formation is measured. Compounds that promote blood vessel formation such as compounds that promote the initiation of angiogenesis or the differentiation processes are determined, and can then be added exogenously or the cells used to engineer the blood vessels can be transfected to express the relevant compound. Inducible expression systems can be used to turn on protein expression by the transfected cells at the relevant time for proper blood vessel development and maturation.

Test compounds can be incorporated in the matrix itself, or delivered locally to the microvascular scaffold by injection or by a drug-releasing matrix. Test compounds can also be administered systemically by oral administration, injection (e.g., intravenous, intraperitoneal, subcutaneous), or by implantation of a pump or device for controlled release

The engineered blood vessels of the invention can also be used as a model for the high-throughput low-cost screening of candidate compounds to identify those that can induce or prevent angiogenesis or vasculogenesis for therapeutic purposes.

Any number of methods are available for carrying out screening assays to identify new candidate compounds that promote or prevent the angiogenesis of the engineered blood vessels. In one working example, candidate compounds are added at varying concentrations directly to the engineered blood vessels or to the matrix or scaffold components. Angiogenesis or vasculogenesis can then be assayed using standard methods such as those described herein (e.g., the methods described in Jain et al., Nat. Rev. Cancer 2:266-276, 2002). The amount of angiogenesis or vasculogenesis in the presence of the candidate compound is compared to that measured in a control vessel not treated with the candidate compound. A compound that promotes angiogenesis or vasculogenesis is considered useful in the invention; and such a compound may be used, for example, as a therapeutic to induce angiogenesis, for example in regenerative medicine or in the treatment of various ischemic diseases. A compound that prevents angiogenesis or vasculogenesis is also considered useful in the invention; and such a compound may be used, for example, as a therapeutic to prevent angiogenesis, for example in the treatment of cancer.

The method of screening may also involve high-throughput techniques employing standard computerized robotic and microtiter plates. In general, the chemical screening methods of the invention provide a straightforward means for selecting synthetic or natural product extracts or compounds of interest from a large population which are further evaluated and condensed to a few active and selective materials. Constituents of this pool are then purified and evaluated in the methods of the invention to determine their ability to modulate angiogenesis or vasculogenesis or both.

Test Extracts and Agents

In general, novel drugs are identified from large libraries of both natural product or synthetic (or semi-synthetic) extracts or chemical libraries according to methods known in the art. The screening methods of the present invention are appropriate and useful for testing agents from a variety of sources for possible activity in vitro. The initial screens may be performed using a diverse library of agents, but the method is suitable for a variety of other compounds and compound libraries. Such compound libraries can be combinatorial libraries, natural product libraries, or other small molecule libraries. In addition, compounds from commercial sources can be tested, as well as commercially available analogs of identified inhibitors.

Virtually any number of chemical extracts or compounds known to those skilled in the art of drug discovery and development can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds, including nucleic-acid ligands such as apatmers. Synthetic compound libraries are commercially available from Nanoscale Combinatorial Synthesis Inc., Mountain View, Calif., ChemDiv Inc., San Diego, Calif., Pharmacopeia Drug Discovery, Princeton, N.J., and ArQule Inc., Medford, Mass. Alternatively, libraries of natural compounds in the form of bacterial, fungal, plant, and animal extracts are commercially available from a number of sources, including Phytera Inc., Worcester, Mass. and Panlabs Inc., Bothell, Wash. In addition, natural and synthetically produced libraries are produced, if desired, according to methods known in the art, e.g., by standard extraction and fractionation methods. Devices for the preparation of combinatorial libraries are also commercially available, for example, Advanced ChemTech, Louisville, Ky. and Argonaut Technologies Inc., San Carlos, Calif. Furthermore, if desired, any library or compound is readily modified using standard chemical, physical, or biochemical methods.

When a crude extract is found to have activity that either promotes or prevents angiogenesis or vasculogenesis in vitro or in vivo, further fractionation of the positive lead extract is necessary to isolate chemical constituents responsible for the observed effect. Thus, the goal of the extraction, fractionation, and purification process is the careful characterization and identification of a chemical entity within the crude extract having activity that modulates angiogenesis or vasculogenesis in vitro or in vivo. Methods of fractionation and purification of such heterogenous extracts are known in the art.

Since many of the compounds that constitute currently available combinatorial and natural products libraries, as well as those found in natural products preparations, are not characterized, the screening methods of this invention provide novel compounds which are active as agonists or antagonists in the particular assays, in addition to identifying known compounds which are active in the screens. Therefore, this invention includes such novel compounds, as well as the use of both novel and known compounds in pharmaceutical compositions and methods of treating diseases or disorders where angiogenesis or vasculogenesis is affected.

Other Embodiments

From the foregoing description, it will be apparent that variations and modifications may be made to the invention described herein to adapt it to various usages and conditions. Such embodiments are also within the scope of the following claims.

All publications mentioned in this specification are herein incorporated by reference to the same extent as if each independent publication was specifically and individually indicated to be incorporated by reference.

What is claimed is:

1. A method for inducing blood vessel formation or engineering blood vessels in a tissue or organ of a mammal, said method comprising administering one or more cells selected from the group consisting of preadipocytes, adipocytes not having a genetic modification, perivascular cells, vascular smooth muscle cells, mesenchymal precursor cells, mesenchymal cells, and fibroblasts to a tissue or organ of a mammal in need of increased blood vessel formation or engineered blood vessels.

2. The method of claim 1, wherein said mammal has a deficiency of at least 5% of a particular cell type.

3. The method of claim 1, wherein said mammal has damage to said tissue or organ, and wherein said administering provides a dose of cells sufficient to increase a biological function of said tissue or organ.

4. The method of claim 1, wherein said mammal has a disease, disorder, or condition, and wherein said administering provides a dose of cells sufficient to ameliorate or stabilize said disease, disorder, or condition.

5. The method of claim 1, wherein said preadipocyte is a 3T3-F442A cell.

6. The method of claim 1, wherein said mesenchymal precursor cell is a 10T1/2 cell.

7. The method of claim 1, further comprising administering to said mammal one or more cells selected from the group consisting of blood vascular endothelial cells, lymph vascular endothelial cells, endothelial cell lines, primary culture endothelial cells, endothelial cells derived from stem cells, bone marrow derived stem cells, cord blood derived cells, HUVEC, lymphatic endothelial cells, and endothelial pregenitor cells.

8. The method of claim 7, wherein said one or more cells is a HUVEC cell.

9. The method of claim 1, further comprising administering a matrix to said mammal.

10. The method of claim 9, wherein said matrix comprises fibronectin.

11. The method of claim 9, wherein said matrix comprises collagen.

12. The method of claim 1, wherein said method increases the number of cells of said tissue or organ by at least 5% compared to a corresponding untreated control tissue or organ.

13. The method of claim 1, wherein said method increases the biological activity of a tissue or organ by at least 5% compared to a corresponding untreated control tissue or organ.

14. The method of claim 1, wherein said method increases blood vessel formation in said tissue or organ by at least 5% compared to a corresponding untreated control tissue or organ.

15. The method of claim 1, wherein said tissue or organ is selected from the group consisting of bladder, bone, brain, breast, cartilage, nervous tissue, esophagus, fallopian tube, heart, pancreas, intestines, gallbladder, kidney, liver, lung, ovaries, prostate, skeletal muscle, skin, spinal cord, spleen, stomach, testes, thymus, thyroid, trachea, urogenital tract, ureter, urethra, and uterus.

16. The method of claim 1, wherein said mammal is a human.

17. The method of claim 1, wherein said cells are part of a microvascular scaffold.

18. A method for increasing blood vessel formation or engineering blood vessels in a tissue or organ, said method comprising administering one or more cells selected from the group consisting of perivascular cells, vascular smooth muscle cells, mesenchymal precursor cells, mesenchymal cells, and fibroblasts to a tissue or organ in need of increased blood vessel formation or engineered blood vessels.

19. The method of claim 18, wherein said mesenchymal precursor cell is a 10T1/2 cell.

20. The method of claim 1, wherein said administering to said tissue or organ is carried out in vivo.

21. The method of claim 1, wherein said administering to said tissue or organ is carried out ex vivo.

22. The method of claim 18, further comprising administering to said tissue or organ one or more cells selected from the group consisting of blood vascular endothelial cells, lymph vascular endothelial cells, endothelial cell lines, primary culture endothelial cells, endothelial cells derived from stem cells, bone marrow derived stem cells, cord blood derived cells, HUVEC, lymphatic endothelial cells, and endothelial pregenitor cells.

23. The method of claim 22, wherein said one or more cell is a HUVEC cell.

24. The method of claim 18, wherein said method further comprising administering a matrix to said tissue or organ.

25. The method of claim 18, wherein said tissue or organ is selected from the group consisting of bladder, bone, brain, breast, cartilage, nervous tissue, esophagus, fallopian tube, heart, pancreas, intestines, gallbladder, kidney, liver, lung, ovaries, prostate, skeletal muscle, skin, spinal cord, spleen, stomach, testes, thymus, thyroid, trachea, urogenital tract, ureter, urethra, and uterus.

26. The method of claim 18, wherein said method increases the number of cells of said tissue or organ by at least 5% compared to a naturally-occurring corresponding control tissue or organ.

27. The method of claim 18, wherein said method increases the biological activity of a tissue or organ by at least 5% compared to a naturally-occurring corresponding control tissue or organ.

28. The method of claim 18, wherein said method increases blood vessel formation in said tissue or organ by at least 5% compared to a naturally-occurring corresponding control tissue or organ.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. The method of claim 18, wherein said administering to said tissue or organ is carried out in vivo.

80. The method of claim 18, wherein said administering to said tissue or organ is carried out ex vivo.


SEQUENCE LISTING (25 sequences)

SEQ ID NO: 1 - DNA, 24 bp, Artificial Sequence
  Primer
atgacagttttcctttcctttgca

SEQ ID NO: 2 - DNA, 24 bp, Artificial Sequence
  Primer
ttcttcgctgccattctgactcac

SEQ ID NO: 3 - DNA, 24 bp, Artificial Sequence
  Primer
catcatggtggtggaacgtaagga

SEQ ID NO: 4 - DNA, 24 bp, Artificial Sequence
  Primer
atgtggcagatcattttcctaact

SEQ ID NO: 5 - DNA, 23 bp, Artificial Sequence
  Primer
tttggctgggatcttgtcttggc

SEQ ID NO: 6 - DNA, 24 bp, Artificial Sequence
  Primer
ccagtagtaccacttgataccgtt

SEQ ID NO: 7 - DNA, 24 bp, Artificial Sequence
  Primer
ctggaagacagctcctcctcgaag

SEQ ID NO: 8 - DNA, 23 bp, Artificial Sequence
  Primer
atgtgtgatgcctttgtgggaac

SEQ ID NO: 9 - DNA, 23 bp, Artificial Sequence
  Primer
taatcaacataaccatatccaat

SEQ ID NO: 10 - DNA, 19 bp, Artificial Sequence
  Primer
tccggatccatgaactttc

SEQ ID NO: 11 - DNA, 18 bp, Artificial Sequence
  Primer
tggctcaccgccttggct

SEQ ID NO: 12 - DNA, 23 bp, Artificial Sequence
  Primer
ggggcaggttctggtattggatg

SEQ ID NO: 13 - DNA, 24 bp, Artificial Sequence
  Primer
cctcgcacagtgcgccagaaaatg

SEQ ID NO: 14 - DNA, 24 bp, Artificial Sequence
  Primer
gctgcttgttgcactgctgcagct

SEQ ID NO: 15 - DNA, 24 bp, Artificial Sequence
  Primer
tctgcattcacattggctgtgttc

SEQ ID NO: 16 - DNA, 24 bp, Artificial Sequence
  Primer
atgcacttgctgtgcttcttgtct

SEQ ID NO: 17 - DNA, 24 bp, Artificial Sequence
  Primer
ctctgtgttataatgtgcagcagc

SEQ ID NO: 18 - DNA, 24 bp, Artificial Sequence
  Primer
cctcatgatgttccatgtgtactt

SEQ ID NO: 19 - DNA, 24 bp, Artificial Sequence
  Primer
tgtctctctagggctgcattgggt

SEQ ID NO: 20 - DNA, 24 bp, Artificial Sequence
  Primer
catgagattggcagtgatttgctt

SEQ ID NO: 21 - DNA, 24 bp, Artificial Sequence
  Primer
tctgcttgtgtactagcagtgacg

SEQ ID NO: 22 - DNA, 25 bp, Artificial Sequence
  Primer
agggactctgtgtggaagtactgtt

SEQ ID NO: 23 - DNA, 24 bp, Artificial Sequence
  Primer
ctggtctggcacagttgagcagtg

SEQ ID NO: 24 - DNA, 24 bp, Artificial Sequence
  Primer
ggcggcgagaacgagaagaaaaag

SEQ ID NO: 25 - DNA, 24 bp, Artificial Sequence
  Primer
cacgttatcagaaatgtaaaccat